(19) United States
(12) Patent Application Publication
(10) Pub. No.: US 2009/0247770 A1
(43) Pub. Date: Oct. 1, 2009

(54) PROCESS FOR MAKING LACTAM TACHYKININ RECEPTOR ANTAGONISTS

(76) Inventors: Kevin R. Campos, Berkeley Heights, NJ (US); Cheng Chen, Plainsboro, NJ (US); Hideaki Ishibashi, Ibaraki (JP); Shinji Kato, Ibaraki (JP); Artis Klapars, Edison, NJ (US); Yoshinori Kohmura, Ibaraki (JP); David J. Pollard, Monroe, NJ (US); Akihiro Takezawa, Ibaraki (JP); Jacob H. Waldman, Westfield, NJ (US); Debra Wallace, Herts (GB); Nobuyoshi Yasuda, Mountainside, NJ (US)

Correspondence Address: MERCK AND CO., INC, P O BOX 2000, RAHWAY, NJ, 07065-0907, US

(21) Appl. No.: 12/309,999

(22) PCT Filed: Aug. 3, 2007
(86) PCT No.: PCT/US2007/017406

§ 371 Date: Feb. 6, 2009

Related U.S. Application Data
(60) Provisional application No. 60/836,640, filed on Aug. 9, 2006.

(51) Int. Cl. C07D 207/24

(52) U.S. Cl. 548/550

(57) ABSTRACT

The present invention is directed to a process for preparing α,α disubstituted γ-lactam derivatives of formula (I) that are useful as neurokinin-1 (NK-1) receptor antagonists, and inhibitors of tachykinin and in particular substance P. The compounds are useful in the treatment of certain disorders, including emesis, urinary incontinence, depression, and anxiety.

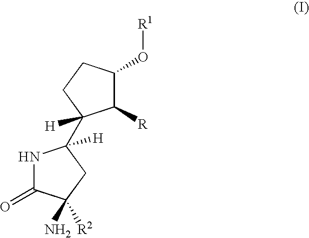

### BACKGROUND OF THE INVENTION

Human neurokinin-1 (hNK-1) is a G-protein-coupled receptor, which is concentrated in the central nervous system and gastrointestinal tissue. See Nicoll, R. A.; Schenker, C.; Leeman, S. E. Annu. Rev. Neurosci. 1980, 3, 227. The neuropeptide substance P (SP) is the preferred ligand for the hNK-1 receptor, and engages in the moderation of many biological processes. See (a) Guard, S.; Watson, S. P. Neurochem. Int. 1991, 18, 149. (b) Takeuchi, Y.; Shands, E. F. B.; Beusen, D. D.; Marshall, G. R. J. Med. Chem. 1998, 41, 3609 and references cited therein. Control of the interaction between SP and hNK-1 has been implicated in the treatment of diverse array of medical disorders including important clinical areas such as depression, anxiety, inflammatory bowel disease, and pain. See (a) Quatara, L.; Maggi, C. A. Neuropeptides 1998, 32, 1. (b) Rupniak, N. M. J.; Kramer, M. S. Trends Pharmacol. Sci. 1999, 20, 485. As a result, there is intense, ongoing pharmaceutical research to identify potent and selective hNK-1 receptor antagonists as potential therapeutic agents. See (a) Owen, S. N.; Seward, E. M.; Swain, C. J.; Williams, B. J. U.S. Pat. No. 6,458,830 B1, 2001. (b) Finke, P. E. MacCoss, M.; Meurer, L. C.; Mills, S. G.; Caldwell, C. G.; Chen, P.; Durette, P. L.; Hale, J.; Holson, E.; Kopka, I.; Robichaud, A. PCT Int. Appl. WO 9714671, 1997. (c) Hale, J. J.; Mills, S. G.; MacCoss, M.; Finke, P. E.; Cascieri, M. A.; Sadowski, S.; Ber, E.; Chicchi, G. G.; Kurtz, M.; Metzger, J.; Elermann, G.; Tsou, N. N.; Tattersall D.; Rupniak, N. M. J.; Williams, A. R.; Rycroft, W.; Hargreaves, R.; MacIntyre, D. E. J. Med. Chem. 1998, 41, 4607.

This application is directed to a process of making certain lactam hNK-1 receptor antagonists. This class of compounds as well an alternative process for making this class of compounds are disclosed in WO2006/002117, published on Jan. 5, 2006 and US 2005-0282886, published Dec. 22, 2005. The present invention is directed to a convergent, stereocontrolled asymmetric synthesis of lactam hNK-1 receptor antagonists.

### SUMMARY OF THE INVENTION

The present invention is directed to a process for preparing certain α, α disubstituted γ-lactam derivatives that are useful as neurokinin-1 NK-1) receptor antagonists, and inhibitors of tachykinin and in particular substance P. The compounds are useful in the treatment of certain disorders, including emesis, urinary incontinence, depression, and anxiety.

### BRIEF DESCRIPTION OF THE DRAWINGS

FIG. 1 is a characteristic X-ray diffraction pattern of the crystalline anhydrous Form I of the benzensulfonate salt of the compound of Formula Ia.

FIG. 2 is a typical DSC curve of the crystalline anhydrous Form I of the benzensulfonate salt of the compound of Formula Ia.

### DETAILED DESCRIPTION OF THE INVENTION

In one aspect the invention encompasses a process of making lactam tachykinin receptor antagonists of Formula I

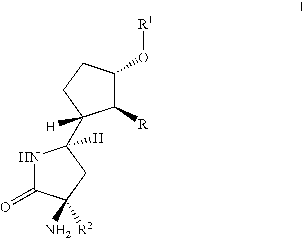

or a pharmaceutically acceptable salt thereof, wherein:
R² is selected from the group consisting of:
(1) hydrogen, and
(2) C₁₋₆alkyl;
   R is selected from the group consisting of:
(1) phenyl, unsubstituted or substituted with one or more of R¹¹, R¹² and R¹³;
(2) C₁₋₈ alkyl, unsubstituted or substituted with one or more of the substituents selected from:
   (a) hydroxy,
   (b) oxo,
   (c) C₁₋₆ alkoxy,
   (d) phenyl-C₁₋₃ alkoxy,
   (e) phenyl,
   (f) —CN,
   (g) halo,
   (h) —NR⁹R¹⁰, wherein R⁹ and R¹⁰ are independently selected from:
      (1) hydrogen,
      (2) C₁₋₆ alkyl,
      (3) hydroxy-C₁₋₆ alkyl, and
      (4) phenyl,
   (i) —NR⁹COR¹⁰, wherein R⁹ and R¹⁰ are as defined above,
   (j) —NR⁹CO₂R¹⁰, wherein R⁹ and R¹⁰ are as defined above,
   (k) —CONR⁹R¹⁰, wherein R⁹ and R¹⁰ are as defined above,
   (l) —COR⁹, wherein R⁹ is as defined above, and
   (m) —CO₂R⁹, wherein R⁹ is as defined above;
(3) C₂₋₆ alkenyl, unsubstituted or substituted with one or more of the substituent(s) selected from:
   (a) hydroxy,
   (b) oxo,
   (c) C₁₋₆ alkoxy,
   is (d) phenyl-C₁₋₃ alkoxy,
   (e) phenyl,
   (f) —CN,
   (g) halo,
   (h) —CONR⁹R¹⁰ wherein R⁹ and R¹⁰ are as defined above,
   (i) —COR⁹ wherein R⁹ is as defined above,
   (j) —CO₂R⁹, wherein R⁹ is as defined above;
(4) heterocycle, wherein the heterocycle is selected from the group consisting of:
   (A) benzimidazolyl,
   (B) benzofuranyl,
   (C) benzothiophenyl,
   (D) benzoxazolyl,
   (E) furanyl,
   (F) imidazolyl,
   (G) indolyl,
   (H) isooxazolyl,
   (I) isothiazolyl,
   (J) oxadiazolyl,
   (K) oxazolyl,
   (L) pyrazinyl,
   (M) pyrazolyl,
   (N) pyridyl,
   (O) pyrimidyl,
   (P) pyrrolyl,
   (Q) quinolyl,
   (R) tetrazolyl,
   (S) thiadiazolyl,
   (T) thiazolyl,
   (U) thienyl,
   (V) triazolyl,
   (W) azetidinyl,
   (X) 1,4-dioxanyl,
   (Y) hexahydroazepinyl,
   (Z) piperazinyl,
   (AA) piperidinyl,
   (AB) pyrrolidinyl,
   (AC) tetrahydrofuranyl, and
   (AD) tetrahydrothienyl,
   and wherein the heterocycle is unsubstituted or substituted with one or more substituent(s) selected from:
      (i) C₁₋₆ alkyl, unsubstituted or substituted with halo, —CF₃, —OCH₃, or phenyl,
      (ii) C₁₋₆ alkoxy,
      (iii) oxo,
      (iv) hydroxy,
      (v) thioxo,
      (vi) —SR⁹, wherein R⁹ is as defined above,
      (vii) halo,
      (viii) cyano,
      (ix) phenyl,
      (x) trifluoromethyl,
      (xi) —(CH₂)ₘ—NR⁹R¹⁰, wherein m is 0, 1 or 2, and R⁹ and R¹⁰ are as defined above,
      (xii) —NR⁹COR¹⁰, wherein R⁹ and R¹⁰ are as defined above,
      (xiii) —CONR⁹R¹⁰, wherein R⁹ and R¹⁰ are as defined above,
      (xiv) —CO₂R⁹, wherein R⁹ is as defined above, and
      (xv) —(CH₂)ₘ—OR⁹, wherein m and R⁹ are as defined above;
         R¹ is selected from the group consisting of:
(1)

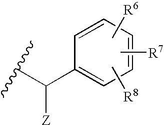

(2) —C₁₋₈ alkyl, wherein alkyl is unsubstituted or substituted with one or more of the substituents selected from:
   (a) hydroxy,
   (b) oxo,
   (c) C₁₋₆ alkoxy,
   (d) phenyl-C₁₋₃ alkoxy,
   (e) phenyl,
   (f) —CN,
   (g) halo,
   (h) —NR⁹R¹⁰, wherein R⁹ and R¹⁰ are as defined above,
   (i) —NR⁹COR¹⁰, wherein R⁹ and R¹⁰ are as defined above,
   (j) —NR⁹CO₂R¹⁰, wherein R⁹ and R¹⁰ are as defined above,
   (k) —CONR⁹R¹⁰, wherein R⁹ and R¹⁰ are as defined above,
   (l) —COR⁹, wherein R⁹ is as defined above, and
   (m) —CO₂R⁹, wherein R⁹ is as defined above;
(3) —C₂₋₆ alkenyl, wherein alkenyl is unsubstituted or substituted with one or more of the substituent(s) selected from:
   (a) hydroxy,
   (b) oxo,
   (c) C₁₋₆ alkoxy,
   (d) phenyl-C₁₋₃ alkoxy,
   (e) phenyl,
   (f) —CN,
   (g) halo,
   (h) —CONR⁹R¹⁰ wherein R⁹ and R¹⁰ are as defined above,
   (i) —COR⁹ wherein R⁹ is as defined above,
   (j) —CO₂R⁹, wherein R⁹ is as defined above;
(4) —(CO)-phenyl, wherein the phenyl is unsubstituted or substituted with one or more of R⁶, R⁷ and R⁸;
   R⁶, R⁷ and R⁸ are independently selected from the group consisting of:
(1) hydrogen;
(2) C₁₋₆ alkyl, unsubstituted or substituted with one or more of the substituents selected from:
   (a) hydroxy,
   (b) oxo,
   (c) C₁₋₆ alkoxy,
   (d) phenyl-C₁₋₃ alkoxy,
   (e) phenyl,
   (f) —CN,
   (g) halo,
   (h) —NR⁹R¹⁰, wherein R⁹ and R¹⁰ are as defined above,
   (i) —NR⁹COR¹⁰, wherein R⁹ and R¹⁰ are as defined above,
   (j) —NR⁹CO₂R¹⁰, wherein R⁹ and R¹⁰ are as defined above,
   (k) —CONR⁹R¹⁰, wherein R⁹ and R¹⁰ are as defined above,
   (l) —COR⁹, wherein R⁹ is as defined above, and
   (m) —CO₂R⁹, wherein R⁹ is as defined above;
(3) C₂₋₆ alkenyl, unsubstituted or substituted with one or more of the substituent(s) selected from:
   (a) hydroxy,
   (b) oxo,
   (c) C₁₋₆ alkoxy,
   (d) phenyl-C₁₋₃ alkoxy,
   (e) phenyl,
   (f) —CN,
   (g) halo,
   (h) —CONR⁹R¹⁰ wherein R⁹ and R¹⁰ are as defined above,
   (i) —COR⁹ wherein R⁹ is as defined above,
   (j) —CO₂R⁹, wherein R⁹ is as defined above;
(4) C₂₋₆ alkynyl;
(5) phenyl, unsubstituted or substituted with one or more of the substituent(s) selected from:
   (a) hydroxy,
   (b) C₁₋₆ alkoxy,
   (c) C₁₋₆alkyl,
   (d) C₂₋₅ alkenyl,
   (e) halo,
   (f) —CN,
   (g) —NO₂,
   (h) —CF₃,
   (i) —(CH₂)ₘ—NR⁹R¹⁰, wherein m, R⁹ and R¹⁰ are as defined above,
   (j) —NR⁹COR¹⁰, wherein R⁹ and R¹⁰ are as defined above,
   (k) —NR⁹CO₂R¹⁰, wherein R⁹ and R¹⁰ are as defined above,
   (l) —CONR⁹R¹⁰, wherein R⁹ and R¹⁰ are as defined above,
   (m) —CO₂NR⁹R¹⁰, wherein R⁹ and R¹⁰ are as defined above,
   (n) —COR⁹, wherein R⁹ is as defined above;
   (o) —CO₂R⁹, wherein R⁹ is as defined above;
(6) halo,
(7) —CN,
(8) —CF₃,
(9) —NO₂,
(10) —SR¹⁴, wherein R¹⁴ is hydrogen or C₁₋₅alkyl,
(11) —SOR¹⁴, wherein R¹⁴ is as defined above,
(12) —SO₂R¹⁴, wherein R¹⁴ is as defined above,
(13) NR⁹COR¹⁰, wherein R⁹ and R¹⁰ are as defined above,
(14) CONR⁹COR¹⁰, wherein R⁹ and R¹⁰ are as defined above,
(15) NR⁹R¹⁰, wherein R⁹ and R¹⁰ are as defined above,
(16) NR⁹CO₂R¹⁰, wherein R⁹ and R¹⁰ are as defined above,
(17) hydroxy,
(18) C₁₋₆alkoxy,
(19) COR⁹, wherein R⁹ is as defined above,
(20) CO₂R⁹, wherein R⁹ is as defined above,
(21) 2-pyridyl,
(22) 3-pyridyl,
(23) 4-pyridyl,
(24) 5-tetrazolyl,
(25) 2-oxazolyl, and
(26) 2-thiazolyl;
   R¹¹, R¹² and R¹³ are independently selected from the definitions of R⁶, R⁷ and R⁸; and
   Z is selected from:
(1) hydrogen,
(2) C₁₋₆ alkyl, and
(3) hydroxyl;
   comprising reacting a compound of Formula A

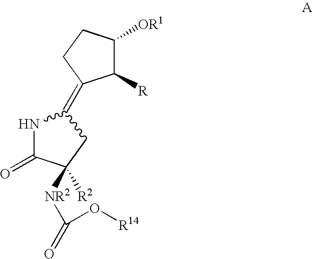

wherein R¹⁴ is selected from R⁶, with a reducing agent under acidic conditions to yield a compound of Formula B

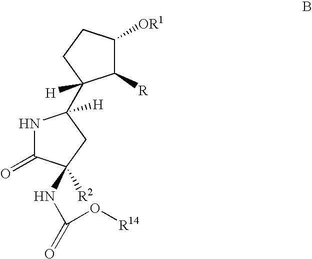

and reacting a compound of Formula B with a strong acid to yield a compound of Formula I, and optionally subsequently forming a pharmaceutically acceptable salt of the compound of Formula I by reacting the compound of Formula I with the corresponding acid of the salt to form the pharmaceutically acceptable salt of the compound of Formula I.

The invention also encompasses the process described above further comprising making the compound of Formula A by reacting a compound of Formula C

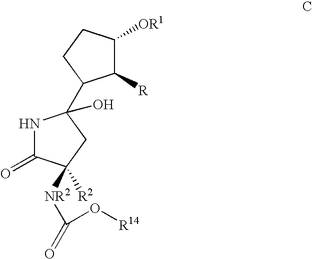

with an acid to make a compound of Formula A.

The invention also encompasses the process described above further comprising making the compound of Formula C by reacting a compound of Formula D

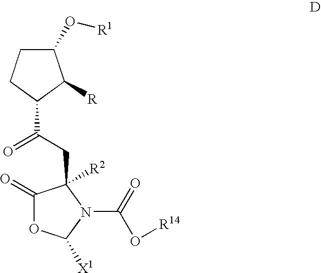

wherein X¹ is C₁₋₆alkyl or phenyl, with ammonia or a salt thereof to yield a compound of Formula C. The invention also encompasses the process described above further comprising making the compound of Formula D by reacting a compound of Formula E

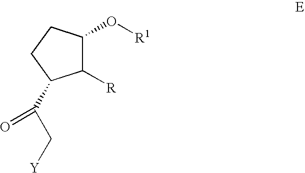

wherein Y is a halogen, with a compound of Formula F

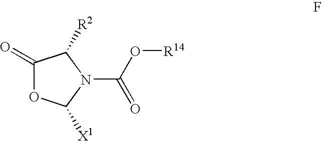

and a metal amide of Formula M¹N(R¹⁵)₂ or M¹N(Si(R¹⁵)₃)₂, wherein M¹ is Li, Na, K or Mg, and each R¹⁵ is independently selected from C₁₋₄alkyl, in a first aprotic organic solvent to yield a compound of Formula D.

The invention also encompasses the process described above further comprising making the compound of Formula E by reacting the compound of Formula G

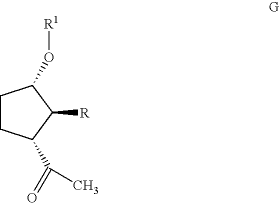

with a halogenating agent to yield a compound of Formula E.

The invention also encompasses the process described above further comprising making the compound of Formula G by reacting the compound of Formula H

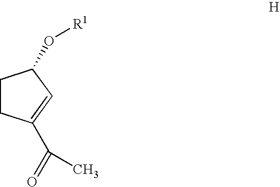

with R-M², wherein M² is a metal, in the presence of a first transition metal catalyst and a Lewis acid, to yield a compound of Formula G.

The invention also encompasses the process described above further comprising making the compound of Formula H by reacting a compound of Formula J

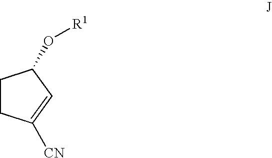

with CH₃Li in tert-butyl methyl ether (MTBE) to yield a compound of Formula H.

The invention also encompasses the process described above further comprising making the compound of Formula J by reacting a compound of Formula K

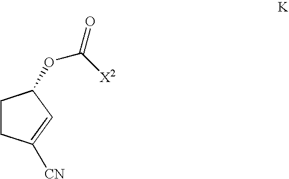

wherein X² is selected from R, with R¹—OH in the presence of a second transition metal catalyst catalyst, a ligand and a zinc additive to yield a compound of Formula J.

The invention also encompasses the process described above further comprising making the compound of Formula K by enzymatically reducing the compound of Formula L

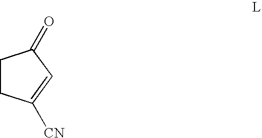

and subsequently reacting the product with X²—COCl to yield a compound of Formula K.

The invention also encompasses the process described above further comprising making the compound of Formula L by reacting a compound of Formula M

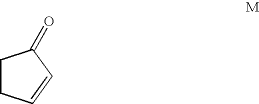

with a brominating agent followed by reaction with a cyanating agent in the presence of a buffer to yield a compound of Formula L.

The invention also encompasses the process described above wherein Y is I and further comprising making the compound of Formula E by reacting a compound of Formula N

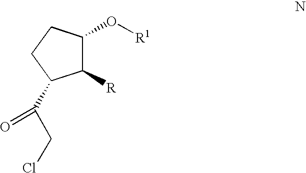

with M³-I, wherein M³ is Li, Na or K, to yield a compound of Formula E.

The invention also encompasses the process described above further comprising making the compound of Formula N by reacting a compound of Formula O

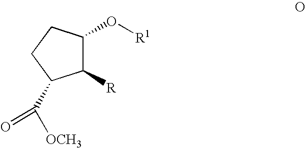

with ClCH₂CO₂H, ClCH₂I, or ClCH₂Br and a metal amide of Formula M⁴N(R¹⁶)₂ or M⁴N(Si(R¹⁶)₃)₂, wherein M⁴ is Li, Na, K, or Mg, and each R¹⁶ is independently selected from C₁₋₄alkyl in a second aprotic organic solvent at a temperature range of about −20° C. to about 40° C. to yield a compound of Formula N.

The invention also encompasses the process described above further comprising making the compound of Formula O by reacting a compound of Formula P

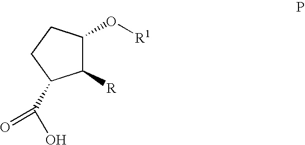

or a triethylamine salt thereof, with a methylating agent to yield a compound of Formula O.

The invention also encompasses the process described above further comprising making the compound of Formula P by reacting a compound of Formula Q

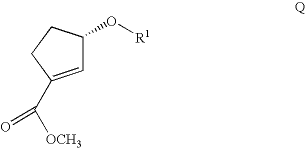

with R-M⁵, wherein M⁵ is M² is a metal, in the presence of a first transition metal catalyst and a Lewis acid, and optionally followed by triethylamide to form the salt, to yield the compound of Formula P or the triethylamine salt thereof.

The invention also encompasses the process described above wherein the compound of Formula Q is made by reacting a compound of Formula R

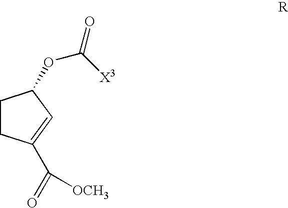

wherein X³ is selected from R, with R¹—OH in the presence of a second transition metal catalyst, a ligand and a zinc additive to yield a compound of Formula Q.

The invention also encompasses the process described above further comprising making the compound of Formula R by enzymatically reducing the compound of Formula S

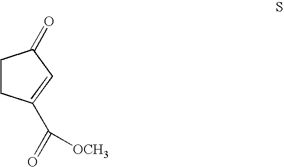

and subsequently reacting the product with X³—COCl to yield a compound of Formula R.

The invention also encompasses the process described above further comprising making the compound of Formula S by reacting a compound of Formula T

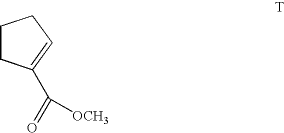

with an oxidizing agent to yield a compound of Formula S.

The invention also encompasses the processes described above wherein R¹ is

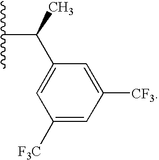

The invention also encompasses the processes described above wherein R is

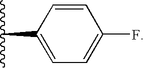

The invention also encompasses the processes described above wherein R² is methyl.

The invention also encompasses the process described above wherein the compound of Formula I is represented by Formula Ia

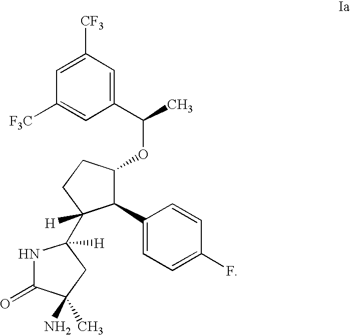

The invention also encompasses the process described above wherein the compound of Formula Ia is a pharmaceutically acceptable salt.

The invention also encompasses the process described above wherein the pharmaceutically acceptable salt is the benzenesulfonate salt and the corresponding acid of the salt is benzenesulfonic acid.

The invention also encompasses the benzenesulfonate salt of the compound of Formula Ia:

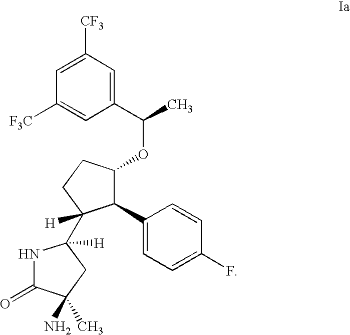

The invention also encompasses the anhydrous crystalline form of this benzenesulfonate salt designated Form I and exhibiting characteristic diffraction peaks corresponding to d-spacings of about 21.2, 9.1, and 8.5 angstroms. Anhydrous Form I is further characterized by the d-spacings of 13.5, 10.9 and 5.5 angstroms. Anhydrous Form I is even further characterized by the d-spacings of 4.5, 4.3, and 4.2 angstroms.

The term “first aprotic organic solvent” and “second aprotic organic solvent” mean for example THF, MTBE, dimethoxyethane, DMF, DMAc and dioaxne.

The term “halogenating agent” means for examples Br₂, I₂ and ICl.

The terms “first transition metal catalyst” and “second transition metal catalyst” mean for example [CODRh(OH)]₂ or CuX or CuX₂ wherein X is Br, Cl or I or a palladium catalyst such as Pd(OAc)₂.

The term “ligand” means for example a phosphine ligan, such as 1,3-bis(diphenylphosphino)propane.

The term “zinc additive” means for example Et₂Zn.

The term “enzymatically reducing” means for example reducing with alcohol dehydrogenase (ADH RE), NADH, glucose and glucose dehydrogenase (GDH 103).

The term “brominating agent” means for example Br2 in the presence of catalytic HBr.

The term “cyanating agent” means for example NaCN and KCN.

The term “buffer” means for example acetic acid and NH₄Cl.

The term “methylating agent” means for example, MeI and M⁶CO₃, wherein M⁶ is Li, Na, K, Ca, or, Cs in polar solvent such as DMF, DMAc, DMSO, acetone at 0˜60° C. or MeOH in the presence of acid catalyst such as H₂SO₄, TsOH, MsOH, or PhSO₃H at ambient temperature to reflux.

The term “reducing agent” means for example (C₁₋₄alkyl)₃SiH.

The term “Lewis acid” means for example TMSCl.

The term “metal” means for example (OH)₂, BF₃, MgBr and Li.

The compounds of Formula I have asymmetric centers and this invention includes all of the optical isomers and mixtures thereof.

In addition compounds with carbon-carbon double bonds may occur in Z- and E-forms with all isomeric forms of the compounds being included in the present invention.

When any variable (e.g., alkyl, aryl, R⁶, R⁷, R⁸, R⁹, R¹⁰, R¹¹, R¹², R¹³, etc.) occurs more than one time in any variable or in Formula I, its definition on each occurrence is independent of its definition at every other occurrence.

As used herein, the term “alkyl” includes those alkyl groups of a designated number of carbon atoms of either a straight, branched, or cyclic configuration. Examples of “alkyl” include methyl, ethyl, propyl, isopropyl, butyl, iso-sec- and tert-butyl, pentyl, hexyl, heptyl, 3-ethylbutyl, cyclopropyl, cyclobutyl, cyclopentyl, cyclohexyl, cycloheptyl, norbornyl, and the like. “Alkoxy” represents an alkyl group of indicated number of carbon atoms attached through an oxygen bridge, such as methoxy, ethoxy, propoxy, butoxy and pentoxy. “Alkenyl” is intended to include hydrocarbon chains of a specified number of carbon atoms of either a straight- or branched-configuration and at least one unsaturation, which may occur at any point along the chain, such as ethenyl, propenyl, butenyl, pentenyl, dimethylpentyl, and the like, and includes E and Z forms, where applicable. “Halogen” or “halo”, as used herein, means fluoro, chloro, bromo and iodo.

The term “aryl” means phenyl or naphthyl either unsubstituted or substituted with one, two or three substituents selected from the group consisting of halo, C₁₋₄alkyl, C₁₋₄-alkoxy, NO₂, CF₃, C₁₋₄-alkylthio, OH, —N(R⁶)₂, —CO₂R⁶, C₁₋₄-perfluoroalkyl, C₃₋₆-perfluorocycloalkyl, and tetrazol-5-yl.

The term “heteroaryl” means an unsubstituted, monosubstituted or disubstituted five or six membered aromatic heterocycle comprising from 1 to 3 heteroatoms selected from the group consisting of O, N and S and wherein the substituents are members selected from the group consisting of —OH, —SH, —C₁₋₄-alkyl, —C₁₋₄-alkoxy, —CF₃, halo, —NO₂, —CO₂R⁹, —N(R⁹R¹⁰) and a fused benzo group.

As will be understood by those skilled in the art, pharmaceutically acceptable salts include, but are not limited to salts with inorganic acids such as hydrochloride, sulfate, phosphate, diphosphate, hydrobromide, and nitrate or salts with an organic acid such as malate, maleate, fumarate, tartrate, succinate, citrate, acetate, lactate, methanesulfonate, p-toluenesulfonate, 2-hydroxyethylsulfonate, pamoate, salicylate and stearate. Similarly, pharmaceutically acceptable cations include, but are not limited to sodium, potassium, calcium, aluminum, lithium and ammonium.

The compounds of the present invention are useful in the prevention and treatment of a wide variety of clinical conditions which are characterized by the presence of an excess of tachykinin, in particular substance P, activity. Thus, for example, an excess of tachykinin, and in particular substance P, activity is implicated in a variety of disorders of the central nervous system. Such disorders include mood disorders, such as depression or more particularly depressive disorders, for example, single episodic or recurrent major depressive disorders and dysthymic disorders, or bipolar disorders, for example, bipolar I disorder, bipolar II disorder and cyclothymic disorder; anxiety disorders, such as panic disorder with or without agoraphobia, agoraphobia without history of panic disorder, specific phobias, for example, specific animal phobias, social phobias, obsessive-compulsive disorder, stress disorders including post-traumatic stress disorder and acute stress disorder, and generalised anxiety disorders; schizophrenia and other psychotic disorders, for example, schizophreniform disorders, schizoaffective disorders, delusional disorders, brief psychotic disorders, shared psychotic disorders and psychotic disorders with delusions or hallucinations; delerium, dementia, and amnestic and other cognitive or neurodegenerative disorders, such as Alzheimer's disease, senile dementia, dementia of the Alzheimer's type, vascular dementia, and other dementias, for example, due to HIV disease, head trauma, Parkinson's disease, Huntington's disease, Pick's disease, Creutzfeldt-Jakob disease, or due to multiple aetiologies; Parkinson's disease and other extra-pyramidal movement disorders such as medication-induced movement disorders, for example, neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremour; substance-related disorders arising from the use of alcohol, amphetamines (or amphetamine-like substances) caffeine, cannabis, cocaine, hallucinogens, inhalants and aerosol propellants, nicotine, opioids, phenylglycidine derivatives, sedatives, hypnotics, and anxiolytics, which substance-related disorders include dependence and abuse, intoxication, withdrawal, intoxication delerium, withdrawal delerium, persisting dementia, psychotic disorders, mood disorders, anxiety disorders, sexual dysfunction and sleep disorders; epilepsy; Down's syndrome; demyelinating diseases such as MS and ALS and other neuropathological disorders such as peripheral neuropathy, for example diabetic and chemotherapy-induced neuropathy, and postherpetic neuralgia, trigeminal neuralgia, segmental or intercostal neuralgia and other neuralgias; and cerebral vascular disorders due to acute or chronic cerebrovascular damage such as cerebral infarction, subarachnoid haemorrhage or cerebral oedema.

Tachykinin, and in particular substance P, activity is also involved in nociception and pain. The compounds of the present invention will therefore be of use in the prevention or treatment of diseases and conditions in which pain predominates, including soft tissue and peripheral damage, such as acute trauma, osteoarthritis, rheumatoid arthritis, musculo-skeletal pain, particularly after trauma, spinal pain, myofascial pain syndromes, headache, episiotomy pain, and burns; deep and visceral pain, such as heart pain, muscle pain, eye pain, orofacial pain, for example, odontalgia, abdominal pain, gynaecological pain, for example, dysmenorrhoea, and labour pain; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, and arachnoiditis; pain associated with carcinoma, often referred to as cancer pain; central nervous system pain, such as pain due to spinal cord or brain stem damage; low back pain; sciatica; ankylosing spondylitis, gout; and scar pain.

Tachykinin, and in particular substance P, antagonists may also be of use in the treatment of respiratory diseases, particularly those associated with excess mucus secretion, such as chronic obstructive airways disease, bronchopneumonia, chronic bronchitis, cystic fibrosis and asthma, adult respiratory distress syndrome, and bronchospasm; inflammatory diseases such as inflammatory bowel disease, psoriasis, fibrositis, osteoarthritis, rheumatoid arthritis, pruritis and sunburn; allergies such as eczema and rhinitis; hypersensitivity disorders such as poison ivy; ophthalmic diseases such as conjunctivitis, vernal conjunctivitis, and the like; ophthalmic conditions associated with cell proliferation such as proliferative vitreoretinopathy; cutaneous diseases such as contact dermatitis, atopic dermatitis, urticaria, and other eczematoid dermatitis. Tachykinin, and in particular substance P, antagonists may also be of use in the treatment of neoplasms, including breast tumours, neuroganglioblastomas and small cell carcinomas such as small cell lung cancer.

Tachykinin, and in particular substance P, antagonists may also be of use in the treatment of gastrointestinal (GI) disorders, including inflammatory disorders and diseases of the GI tract such as gastritis, gastroduodenal ulcers, gastric carcinomas, gastric lymphomas, disorders associated with the neuronal control of viscera, ulcerative colitis, Crohn's disease, irritable bowel syndrome and emesis, including acute, delayed or anticipatory emesis such as emesis induced by chemotherapy, radiation, toxins, viral or bacterial infections, pregnancy, vestibular disorders, for example, motion sickness, vertigo, dizziness and Meniere's disease, surgery, migraine, variations in intercranial pressure, gastro-oesophageal reflux disease, acid indigestion, over indulgence in food or drink, acid stomach, waterbrash or regurgitation, heartburn, for example, episodic, nocturnal or meal-induced heartburn, and dyspepsia.

Tachykinin, and in particular substance P, antagonists may also be of use in the treatment of a variety of other conditions including stress related somatic disorders; reflex sympathetic dystrophy such as shoulder/hand syndrome; adverse immunological reactions such as rejection of transplanted tissues and disorders related to immune enhancement or suppression such as systemic lupus erythematosus; plasma extravasation resulting from cytokine chemotherapy, disorders of bladder function such as cystitis, bladder detrusor hyper-reflexia, frequent urination and urinary incontinence, including the prevention or treatment of overactive bladder with symptoms of urge urinary incontinence, urgency, and frequency; fibrosing and collagen diseases such as scleroderma and eosinophilic fascioliasis; disorders of blood flow caused by vasodilation and vasospastic diseases such as angina, vascular headache, migraine and Reynaud's disease; and pain or nociception attributable to or associated with any of the foregoing conditions, especially the transmission of pain in migraine. The compounds of the present invention are also of value in the treatment of a combination of the above conditions, in particular in the treatment of combined post-operative pain and post-operative nausea and vomiting.

The compounds of the present invention are particularly useful in the prevention or treatment of emesis, including acute, delayed or anticipatory emesis, such as emesis induced by chemotherapy, radiation, toxins, pregnancy, vestibular disorders, motion, surgery, migraine, and variations in intercranial pressure. For example, the compounds of the present invention are of use optionally in combination with other antiemetic agents for the prevention of acute and delayed nausea and vomiting associated with initial and repeat courses of moderate or highly emetogenic cancer chemotherapy, including high-dose cisplatin. Most especially, the compounds of the present invention are of use in the treatment of emesis induced by antineoplastic (cytotoxic) agents, including those routinely used in cancer chemotherapy, and emesis induced by other pharmacological agents, for example, rolipram. Examples of such chemotherapeutic agents include alkylating agents, for example, ethyleneimine compounds, alkyl sulphonates and other compounds with an alkylating action such as nitrosoureas, cisplatin and dacarbazine; antimetabolites, for example, folic acid, purine or pyrimidine antagonists; mitotic inhibitors, for example, vinca alkaloids and derivatives of podophyllotoxin; and cytotoxic antibiotics. Particular examples of chemotherapeutic agents are described, for instance, by D. J. Stewart in Nausea and Vomiting: Recent Research and Clinical Advances, Eds. J. Kucharczyk et al, CRC Press Inc., Boca Raton, Fla., USA (1991) pages 177-203, especially page 188 Commonly used chemotherapeutic agents include cisplatin, dacarbazine (DTIC), dactinomycin, mechlorethamine, streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin and chlorambucil [R. J. Gralla et al in Cancer Treatment Reports (1984) 68(1), 163-172]. A further aspect of the present invention comprises the use of a compound of the present invention for achieving a chronobiologic (circadian rhythm phase-shifting) effect and alleviating circadian rhythm disorders in a mammal.

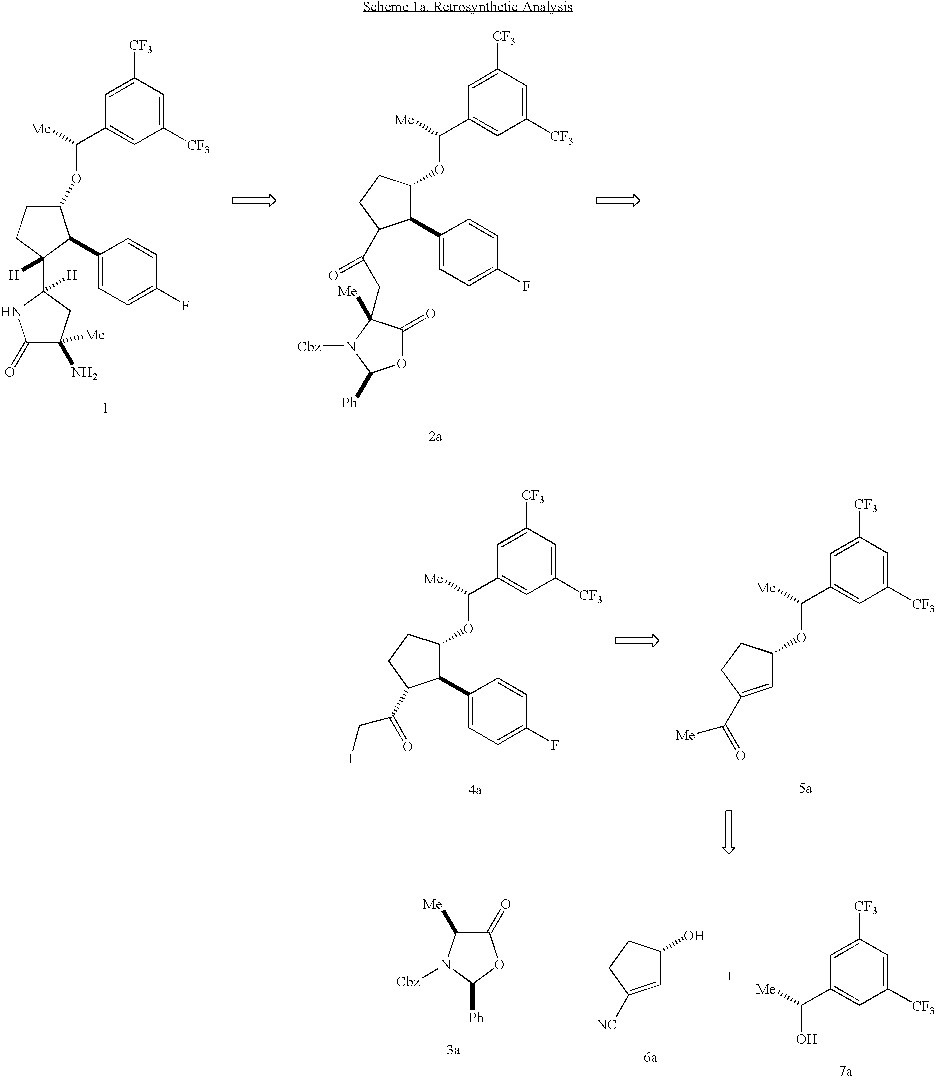

The structural complexity of 1 could be divided into three distinct synthetic challenges: 1) the sterically congested ether which contained stereochemistry at both secondary stereogenic termini, 2) the trans, trans-1,2,3-trisubstituted cyclopentane core, and 3) the pyrrolidinone ring containing two stereogenic centers, one of which was a tertiary amine (Scheme 1a). In order to address the stereochemistry of the remote tertiary amine, we devised a strategy to produce 1 from ketone 2a, which would arise from diastereoselective alkylation of oxazolidinone 3a with iodoketone 4a. The most effective method to control the relative stereochemistry of the cyclopentane core in 4a would be via substrate-controlled conjugate addition of an aryl-metal species on allylic ether 5a, followed by isomerization of the ketone to the thermodynamically-favored diastereomer. We envisioned that the best way to address both secondary stereogenic centers in allylic ether 5a would be via convergent, stereospecific coupling of allylic alcohol 6a and alcohol 7a, each in enantiomerically pure form.

Alcohol 7a is a common structural motif that exists in several hNK-1 antagonists, see (a) Owen, S. N.; Seward, E. M.; Swain, C. J.; Williams, B. J. U.S. Pat. No. 6,458,830 B1, 2001. (b) Finke, P. E. MacCoss, M.; Meurer, L. C.; Mills, S. G.; Caldwell, C. G.; Chen, P.; Durette, P. L.; Hale, J.; Holson, E.; Kopka, I.; Robichaud, A. PCT Int. Appl. WO 9714671, 1997. (c) Hale, J. J.; Mills, S. G.; MacCoss, M.; Finke, P. E.; Cascieri, M. A.; Sadowski, S.; Ber, E.; Chicchi, G. G.; Kurtz, M.; Metzger, J.; Elermann, G.; Tsou, N. N.; Tattersall D.; Rupniak, N. M. J.; Williams, A. R.; Rycroft, W.; Hargreaves, R.; MacIntyre, D. E. J. Med. Chem. 1998, 41, 4607, and is readily available via asymmetric reduction of the corresponding aryl methyl ketone. See Hansen, K. B.; Chilenski, J. R.; Desmond, R.; Devine, P. N.; Grabowski, E. J. J.; Heid, R.; Kubryk, M.; Mathrem D. J.; Varsolona, R. Tetrahedron: Asymmetry 2003, 14, 3581.

In contrast, the enantioselective synthesis of allylic alcohol 6a has not been reported. Application of existing methodologies to the asymmetric reduction of 3-cyanocyclopentenone (8a) afforded 6a in variable yields and moderate enantioselectivities. See (a) Ohkuma, T.; Koizumi, M.; Doucet, H.; Pham, T.; Kozawa, M.; Murata, K.; Katayama, E.; Yokozawa, T.; Ikariya, T.; Noyori, R. J. Am. Chem. Soc. 1998, 120, 13529. (b) Corey, E. J.; Guzman-Perez, A.; Lazerwith, S. E. J. Am. Chem. Soc. 1997, 119, 11769. (c) Yun, J.; Buchwald, S. L.; J. Am. Chem. Soc. 1999, 121, 5640. (d) Brown, H. C.; Ramachandran, P. V. Acc. Chem. Res. 1992, 25, 16-24. (e) Midland, M. M.; Tramontano, A.; Kazbubski, A.; Graham, R. S.; Tsai, D. J. S.; Cardin, D. Tetrahedron 1984, 40, 1371. (f) Noyori, R.; Suzuki, M. Angew. Chem. Int. Ed. Engl. 1984, 23, 847. Although there was no precedent for the asymmetric, biocatalytic reduction of enones similar to 8a, see (a) Fonteneau, L.; Rosa, S.; Buisson, D. Tetrahedron: Asymmetry, 2002, 13, 579. (b) Attolini, M.; Bouguir, F.; Iacazio, G.; Peiffer, G.; Maffei, M. Tetrahedron, 2001, 57, 537, a ketoreductase library was screened. We discovered that alcohol dehydrogenase from Rhodococcus erythropolis (ADH RE) efficiently reduced enone 8a to (S)-allylic alcohol 6a in high yield (93%) and excellent enantioselectivity (>99% ee) (Scheme 2a).

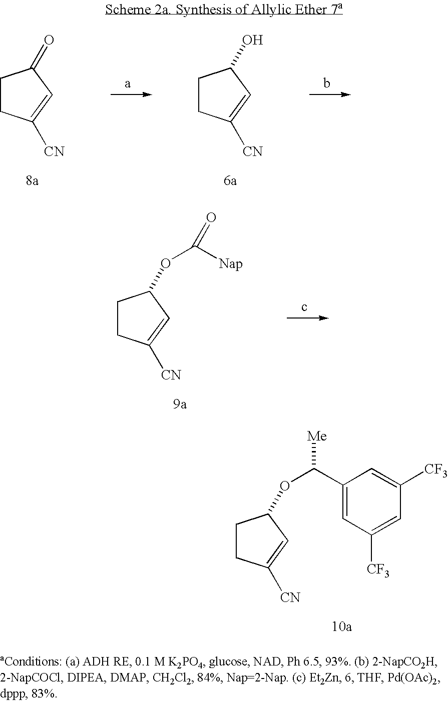

With alcohols 6a and 7a prepared in high optical purity, we sought a method to couple these partners without epimerization at either center. The documented stereospecificity of transformations which proceed via η³-allylmetal intermediates made the Pd-catalyzed allylic etherification an attractive choice; however, alcohol 7a was an unlikely candidate to participate in this coupling due to its steric congestion and poor nucleophilicity. See Kim, H.; Lee. C. Org. Lett. 2002, 4369 and references cited therein. We were pleased to find that, under optimized conditions, a stoichiometric ratio of allylic naphthoate ester 9a and alcohol 6a were coupled using Pd(OAc)₂ and dppp in the presence of 0.5 equivalents of Et₂Zn to afford allylic ether 10a in 83% assay yield and complete retention of configuration at both stereogenic centers.

Although attempts to accomplish a cuprate conjugate addition on the nitrile were unsuccessful, we were able to demonstrate a ligandless Rh-catalyzed conjugate addition (3 mol % [CODRh(OH)₂], EtOH, reflux) using 5.0 equivalents of arylboronic acid or 1.5 equivalents aryl trifluoroborate (K salt). See (a) Batey, R. A.; Thadani, A. N.; Smil, D. V. Org. Letters 1999, 1683. (b) Sakai, M.; Hayashi, H.; Miyaura, N. Organometallics 1997, 16, 4229. Both procedures afforded 11a in 93% assay yield and high diastereoselectivity (>99:1 β-center, 90:10 α-center) after isomerization of the ketone to the thermodynamically-favored diastereomer (NaOMe/MeOH). The nitrile was readily converted to the methyl ketone via treatment with MeLi in MTBE, delivering 12a in 90% assay yield (Scheme 3a).

Alternatively, methyl ketone 13a was readily produced from nitrile 10a (MeLi, MTBE), and as expected, Cu-catalyzed conjugate addition of aryl Grignard delivered 12a in excellent yield (95%) and exceptional diastereoselectivity (>99:1-center, 98:2 β-center) after isomerization of the ketone to the thermodynamically-preferred diastereomer (NaOMe/MeOH). Selective iodination of 13a with ICl in MeOH produced iodoketone 4a in 90% isolated yield. An X-ray crystal structure of 4a verified both the relative and absolute chemistry of the four stereogenic centers assembled through this process.

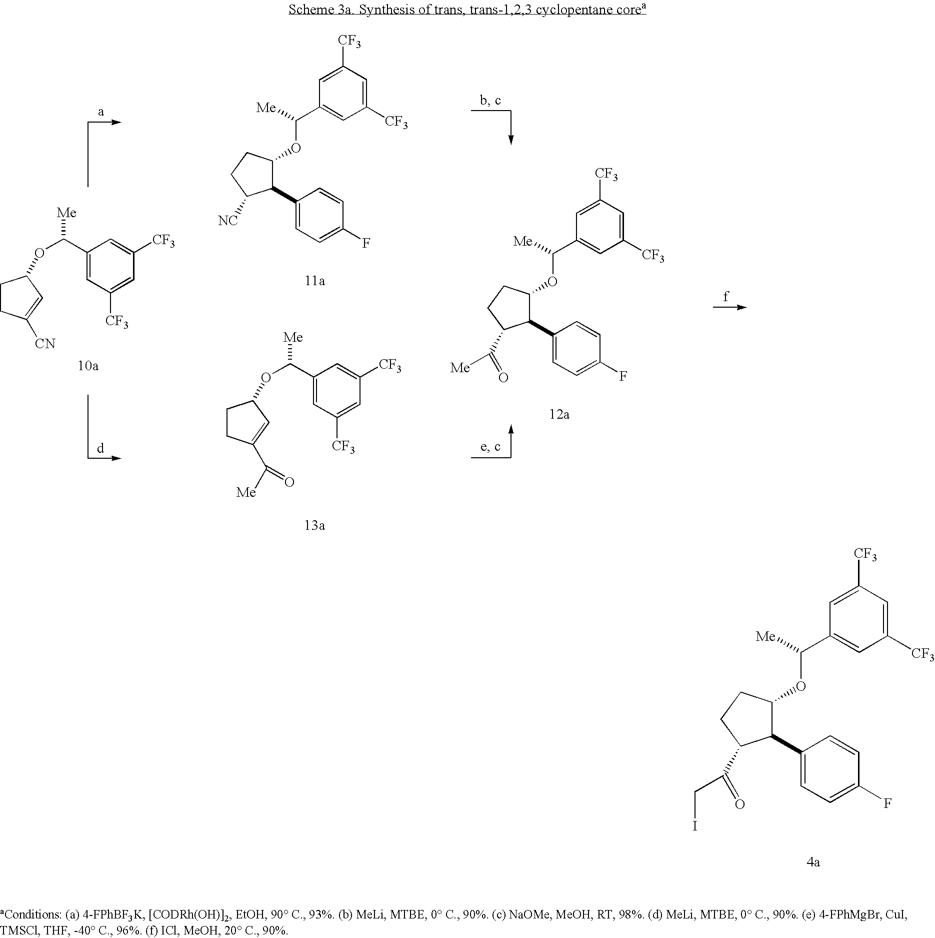

With a convergent and highly selective process to assemble iodoketone (6 steps, 58% yield), we focused our attention on the development of a stereocontrolled method to introduce the pyrrolidinone ring. Alkylation of oxazolidinone 3a with iodoketone 4a afforded 2 in an only modest yield under published methods. See (a) Karady, S.; Amato, J.; Weinstock, L. Tetrahedron Lett. 1984, 25, 4337. (b) Szumigala, Jr., R. H.; Onofiok, E.; Karady, S.; Armstrong, III, J. D.; Miller R. A. Tetrahedron Lett. 2005, 46, 4403. The best result of 90% yield with >99:1 diastereoselectivity was accomplished with 2.4 equivalents of 3 and 2.5 equivalents of LHMDS in toluene/DMPU at low temperature. Cleavage of the oxazolidinone with ammonium hydroxide cleanly delivered diastereomer mixture of animals 14a,b which was dehydrated with methanesulfonic acid to a diastereo-mixture of enamides 15a,b (˜3:1) in 94% yield, prior to the silane reduction because direct reduction from 14 to 16 generates 1 equivalent of water which disturbs Et₃SiH reduction. Enamides 15a,b are in equilibrium through acyliminium cations 17 and 18, because either isolated diastereomerically pure isomer, 15a or 15b, was converted to the same 3:1 mixture of 15a,b under acidic conditions. And acyliminium 18 is thermodynamically more stable than 17. Hence, reduction of 15a,b with Et₃SiH/MeSO₃H predominantly proceeded through 18 and afforded 16 in excellent yield as a 90:10 mixture of diastereomers, together with a little amount of an epimer on the cyclopentane ring via 17. Chemoselective deprotection of 16 was accomplished with HBr/AcOH. Candidate 1 was obtained in 85% yield as a benzenesulfonate.

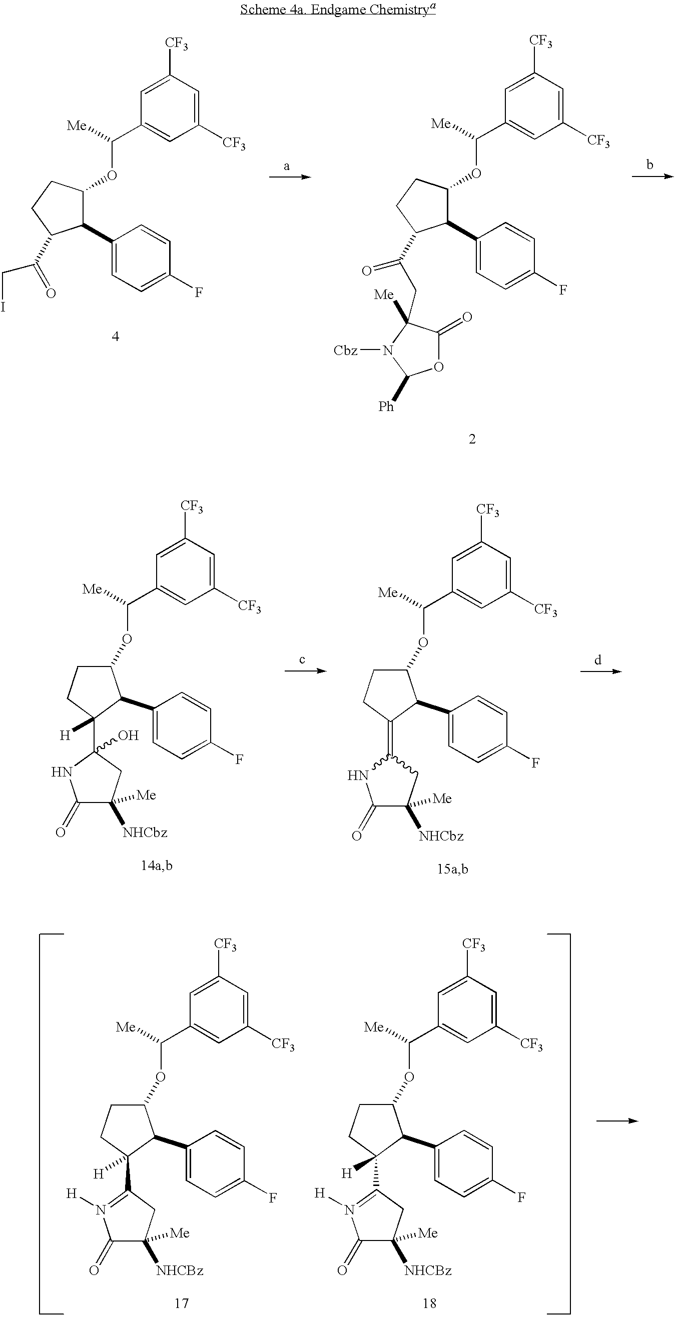

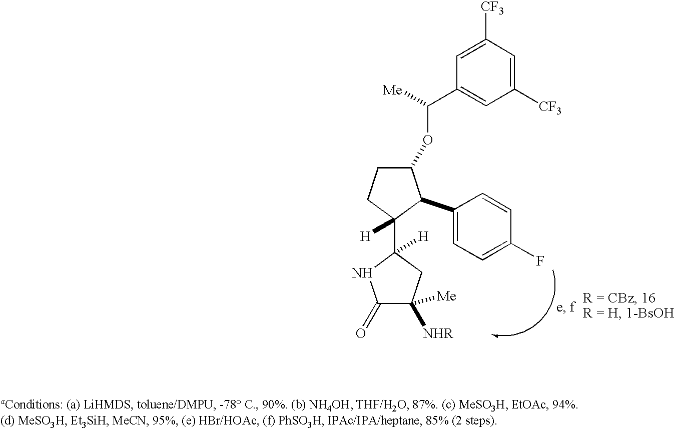

In conclusion, a convergent, highly selective route has been developed for the synthesis of the potent hNK-1 receptor antagonist 1. All 6 stereogenic centers were crafted with outstanding selectivity in a total of 11 steps (23% yield), and the process was used to produce 7 kg of 1. The application of Pd-catalyzed etherification followed by substrate-controlled conjugate addition in cyclic substrates such as 10a and 13a has general application to the stereocontrolled synthesis of highly functionalized cyclopentanoids.

The methodology described above was applied to a variety of systems as was found to have broad application as exemplified by the tables that follow.

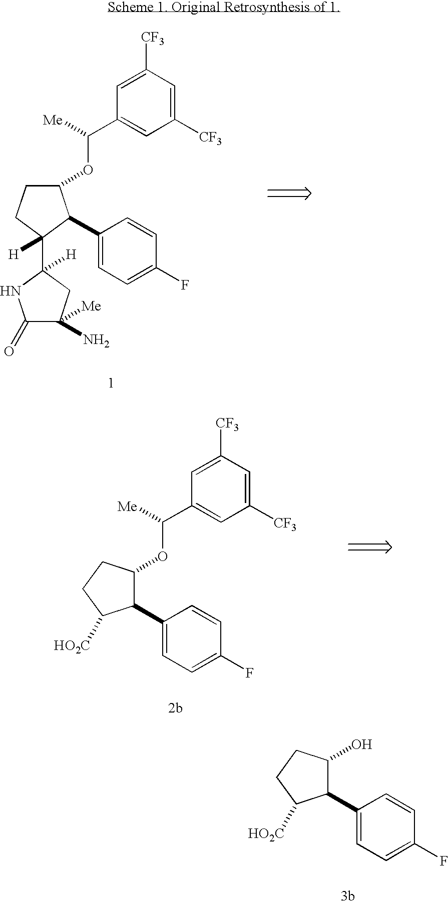

In devising a practical synthesis of 2b, two distinct synthetic challenges must be addressed: 1) the sterically congested ether, which contains stereochemistry at both secondary stereogenic termini and 2) the trans, trans-1,2,3-trisubstituted cyclopentane core. We envisioned that the trans, trans-configuration in 2b, could be effectively assembled via substrate-controlled conjugate addition of an aryl-metal species on allylic ether 4b, followed by equilibration of the ester to the thermodynamically preferred diastereomer (Scheme 2). The most attractive and convergent method for the construction of 4b, would be via stereospecific coupling of allylic alcohol 5b with alcohol 6b, each in enantiomerically pure form. The retrosynthesis described above dissects the target structure into three components of similar size and complexity, which we envisioned to be applicable not only to 2b but also to a range of structural analogs.

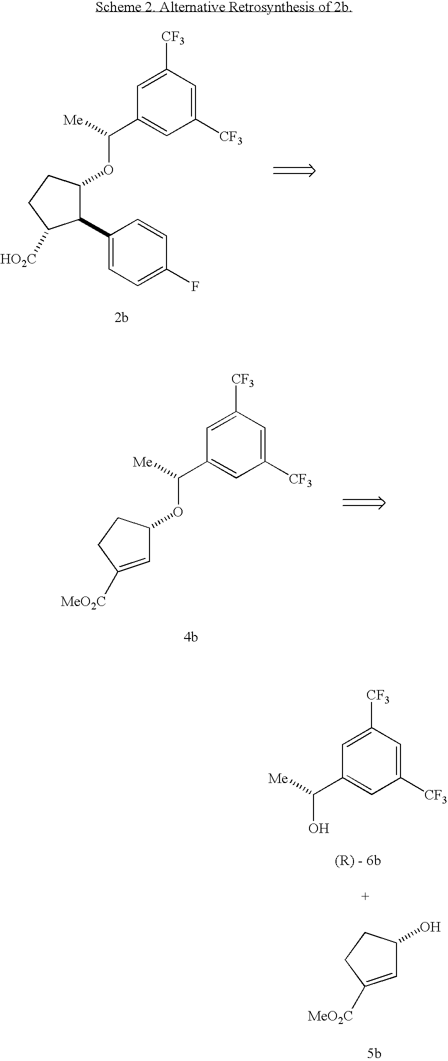

Alcohol 6b is a common structural element that is present in several drug candidates, see a) Nelson, T. D.; Rosen, J. D.; Smitrovich, J. H.; Payack, J.; Craig, B.; Matty, L.; Huffman, M. A.; McNamara, J. Org. Lett. 2005, 55. b) Zhao, M. M.; McNamara, J. M.; Ho, G.-J.; Emerson, K. M.; Song, Z. J.; Tschaen, D. M.; Brands, K. M. J.; Dolling, U.-H.; Grabowski, E. J. J.; Reider, P. J.; Cottrell, I. F.; Ashwood, M. S.; Bishop, B. C. J. Org. Chem. 2002, 6743, and was readily prepared via asymmetric reduction of the corresponding aryl methyl ketone. See Hansen, K. B.; Chilenski, J. R.; Desmond, R.; Devine, P. N.; Grabowski, E. J. J.; Heid, R.; Kubryk, M.; Mathre, D.; Varsolona, R. Tetrahedron: Asymmetry 2003, 3581. In contrast, the enantioselective synthesis of allylic alcohol 5b and structural analogs has not been reported. The most direct route to 5b would be via asymmetric reduction of 3-carboxymethylcyclopentenone (7b). See a) Catino, A. J.; Forslund, R. E.; Doyle, M. P. J. Am. Chem. Soc. 2004, 13622-13623. b) Yu, J-Q.; Corey, E. J. J. Am. Chem. Soc. 2003, 3232-3233. However, application of existing methodologies to the asymmetric reduction of 7b delivered allylic alcohol 6b in moderate yields and mediocre enantioselectivities (Table 1). See a) Ohkuma, T.; Koizumi, M.; Doucet, H.; Pham, T.; Kozawa, M.; Murata, K.; Katayama, E.; Yokozawa, T.; Ikariya, T.; Noyori, R. J. Am. Chem. Soc. 1998, 120, 13529. b) Corey, E. J.; Guzman-Perez, A.; Lazerwith, S. E. J. Am. Chem. Soc. 1997, 119, 11769. c) Yun, J.; Buchwald, S. L.; J. Am. Chem. Soc. 1999, 121, 5640. d) Brown, H. C.; Ramachandran, P. V. Acc. Chem. Res. 1992, 25, 16-24. e) Midland, M. M.; Tramontano, A.; Kazbubski, A.; Graham, R. S.; Tsai, D. J. S.; Cardin, D. Tetrahedron 1984, 40, 1371. f) Noyori, R.; Suzuki, M. Angew. Chem. Int. Ed. Engl. 1984, 23, 847.

In order to determine the viability of a biocatalytic reduction of 7b, see a) Fonteneau, L.; Rosa, S.; Buisson, D. Tetrahedron: Asymmetry, 2002, 13, 579. b) Attolini, M.; Bouguir, F.; Iacazio, G.; Peiffer, G.; Maffei, M. Tetrahedron, 2001, 57, 537, a ketoreductase library screen was performed. We were pleased to find that the alcohol dehydrogenase from Rhodococcus erythropolis (ADH RE) efficiently reduced 7b to 5b in good yield (83%) and excellent enantioselectivity (>99% ee) for the desired (S)-enantiomer (Table 1, entry 4). As testament to the robustness of this process, 3-cyanocyclopentenone (8b), which exhibited extremely poor performance in the Ru-catalyzed transfer hydrogenation, was reduced to allylic alcohol 9b in high yield and excellent enantioselectivity.

TABLE 1
Chemo-and Enantioselective Reduction of 7b.

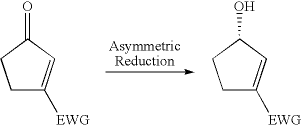

| Entry | EWG | Conditions | % Yield | % ee |
|---|---|---|---|---|
| 1 | CO₂Me (7b) | Ru-cat. NEt₃/HCO₂H^{a} | 90 (5b) | 75 |
| 2 | CO₂Me | (R)-OAB/BH₃-DMS | 75 | 30 |
| 3 | CO₂Me | ADH RE, NADH, FDH | 83 | >99 |
| 4 | CN (8b) | Ru-cat. NEt₃/HCO₂H | 30 (9b) | 57 |
| 5 | CN | ADH RE, NADH, FDH | 92 | >99 |
| 6 | SO₂Ph | ADH RE, NADH, FDH | XX | >99 |
| 7 | NO₂ | ADH RE, NADH, FDH | XX | XX |

^{a}) Ru cat. = [p-cymeneRuCl₂]₂, (S,S)-TsDPEN.

With optically pure allylic alcohol 5b in hand, we sought a method to couple 5b to alcohol 6b without scrambling of either stereogenic center. The documented stereospecificity of reactions which proceed via η3-allyl metal intermediates made the Pd-catalyzed allylic etherification an attractive choice. See a) Shu, C.; Hartwig, J. F. Angew. Chem. Int. Ed. 2004, 4794. b) Kim, H.; Lee. C. Org. Lett. 2002, 4369. b) Evans, P. A.; Leahy, D. K. J. Am. Chem. Soc. 2002, 7882. However, alcohol 6b was an unlikely candidate to participate in this coupling due to its steric congestion and poor nucleophilicity. To our surprise, the zinc alkoxide of 6b readily coupled to allylic acetate 10b under the standard conditions that have been reported for the Pd-catalyzed etherification, delivering allylic ether 4 in 50% yield as a single diastereomer. When 10b, of different levels of enantiomeric excess, was subjected to the etherification with optically pure 6b, the respective amount of the ether diastereomer 4ab was observed, indicating a high degree of stereospecificity (Table 2).

TABLE 2
Pd-Catalyzed Etherification of 9.

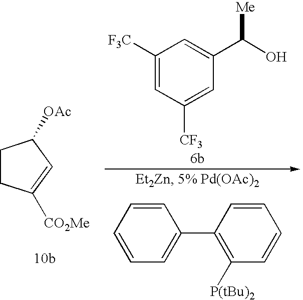

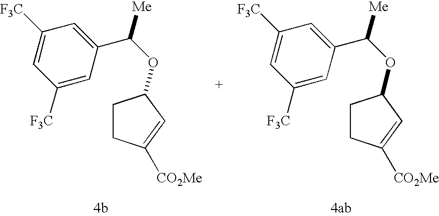

| % ee 10b | % Assay yield 4b+4ab | 4b:4ab |
|---|---|---|
| 0 | 50 | 50:50 |
| 75 | 50 | 87:12 |
| >99 | 50 | >99.5:0.5 |

The moderate yield in the etherification was the result of an extremely reactive allylic ester, which underwent decomposition under the reaction conditions. Attenuation of the reactivity was achieved through the choice of a poorer leaving group, such as benzoate or naphthoate, which improved the yield of 4b to 73% yield. Naphthoate allylic ester (11b) was a crystalline solid, and was chosen for further development. An extensive screening of ligands revealed that although the Buchwald biaryl diphosphines were effective in the Pd-catalyzed etherification, equivalent results could be obtained with 1,3-diphenylphosphinopropane (dppp), which was considerably less expensive and more readily available. As a further improvement, the amount of alcohol 6b could be reduced to 1.0 equivalent with no effect on the assay yield of product. Under optimized conditions, allylic ether 4b was prepared in 80% yield and >99:1 diastereomeric ratio (Scheme 3).

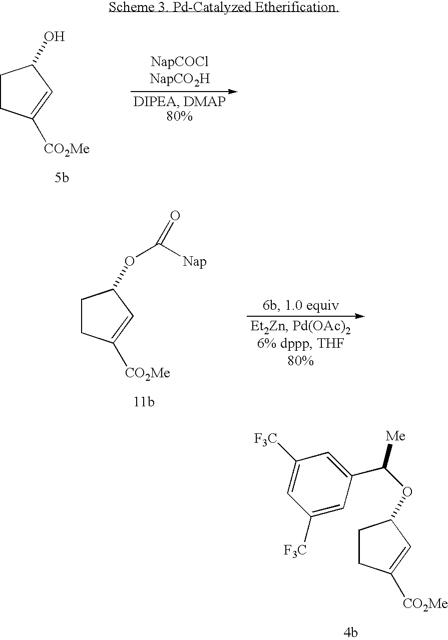

The conditions developed for the Pd-catalyzed allylic etherification were tolerant of a variety of alcohols, providing a diverse array of allylic ethers with complete retention of stereochemistry at the reacting center (Table 3). In the coupling of allylic ester 11b with both enantiomers of alcohol 6b, only a modest difference in reactivity was observed; however in the case of more sterically encumbered alcohols, a clear “matched” and “mismatched” pairing was observed. Additionally, the coupling of allylic esters with achiral alcohols provided the expected allylic ethers in high enantiomeric ratios. The reaction was tolerant of a variety of electron withdrawing groups including esters, nitrites, and even ketones; however cyclic allylic esters lacking an electron withdrawing group at the 3-position were inactive in the allylic etherification.

TABLE 3
Pd-catalyzed etherification of 10b.

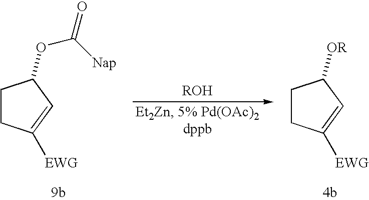

| Entry | EWG | R | % Assay yield (% dr)^{a} |
|---|---|---|---|
| 1 | CO₂Me (11b) | (R)-6b | 78 (>99:1) |
| 2 | CO₂Me | (S)-6b | 65 (>99:1) |
| 3 | CN (12b) | (R)-6b | 80 (>99:1) |
| 4 | CN | (1S,2R,5S)-menthol | 38 (>99:1) |
| 5 | CN | (1R,2S,5R)-menthol | 75 (>99:1) |
| 6 | CN | c-HexOH | 76 (98% ee) |
| 7 | CN | 2-BrPhCH₂OH | 85% (97% ee) |
| 8 | CN | 3-(NO₂)PhCH₂OH | 85% (98% ee) |
| 9 | CN | CF₃CH₂OH | 74% (99% ee) |
| 10 | CN | PhOH | 75 (>99% ee) |
| 11 | CN | 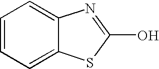 | 67% (94% ee) |
| 12 | C(O)Me | (R)-6b | 55 (99:1) |
| | (13b) | | |

The highly convergent and efficient preparation of allylic ether 4b provided a means to access cyclopentane 2b via diastereoselective conjugate addition of an aryl-metal species. Attempts to accomplish the conjugate addition via Grignard or cuprate methodology led to varying amounts of the desired conjugate addition product along with significant quantities of the 1,2-addition product. Recent advances in enantioselective Rh-catalyzed conjugate additions of aryl boronic acid derivatives led us to attempt this methodology on our substrate. See Hayashi, T.; Yamasaki, K. Chem Rev. 2003, 2829 and references cited therein. Typically, the problem associated with the enantioselective variant is the high rate of reaction for the “ligandless” background reaction. As a result, we subjected substrate 4b to standard conditions (5 equiv boronic acid, 3 mol % [CODRh(OH)]2, dioxane/water) in the absence of ligand. We were pleased to observe 89% assay yield of the desired conjugate addition product with complete stereocontrol. A significant reduction in the amount of the boronic acid could be achieved by employing the aryltrifluoroborate potassium salt, see a) Batey, R.; Thanadi, A. N.; Smil, D. V. Org. Lett. 1999, 1683. b) Darses, S.; Genet, J. P.; Brayer, J. L.; Demoute, J. P. Tetrahedron Lett. 1997, 4393, which required only 1.5 equivalents to achieve complete conversion. We also discovered that ethanol was an excellent alternative to dioxane/water as the solvent medium, providing faster conversion, better yield, and cleaner reactions. Under optimized conditions, 15b was obtained in 93% yield as a 95:5 mixture of epimers after equilibration to the thermodynamically preferred isomer. This mixture was readily hydrolyzed to acid 2b and isolated as the triethylamine salt in excellent yield with good rejection of the undesired epimer (Scheme 4).

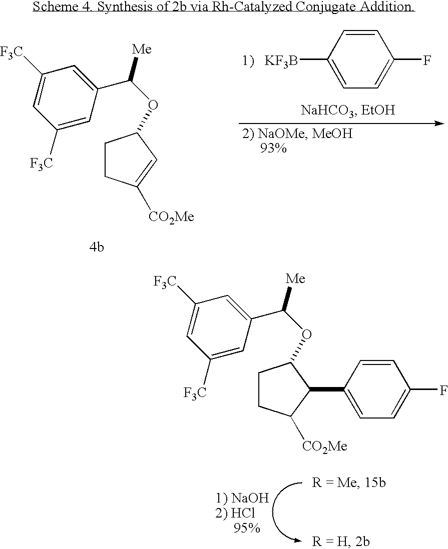

Having demonstrated an efficient process for the production of 2b, we sought to determine the range and scope of the method. A variety of electron-rich and electron-poor aryl boronic acids were suitable nucleophiles in the diastereoselective Rh-catalyzed conjugate addition, providing high yields and complete diastereoselectivity in each case (Table 4). The methodology was tolerant of moderately hindered aryl boronic acids; however, 2,6-disubstituted aryl boronic acids provided low yields of the conjugate addition product. Heterocyclic boronic acids and vinyl boronic acids were completely ineffective in the Rh-catalyzed process, affording <5% of the desired conjugate addition products in every case. This methodology was also effective for α,β-unsaturated ketones and nitriles, which delivered the respective products in excellent yield and diastereoselectivity. All substrates (ester, nitrile, and ketone) gave high epimeric ratios after thermodynamic equilibration (95:5, 92:8, and 98:2 respectively).

TABLE 4
Diastereoselective conjugate additions.

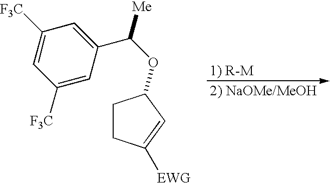

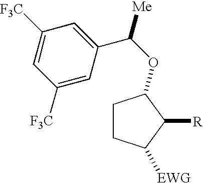

| Entry | EWG | Ar | % yield (dr) |
|---|---|---|---|
| 1 | CO₂Me | 4-FPhB(OH)₂ | 89 (95:5) |
| 2 | CO₂Me | 4-MeOPhB(OH)₂ | 78 (95:5) |
| 3 | CO₂Me | PhB(OH)₂ | 99 (95:5) |
| 4 | CO₂Me | 4-CNPhB(OH)₂ | 84 (94:6) |
| 5 | CO₂Me | 2-MePhB(OH)₂ | 85 (96:4) |
| 6 | CO₂Me | 4-FPhBF₃K | 93 (95:5) |
| 7 | CN | 4-FPhBF₃K | 85 (92:8) |
| 8 | C(O)Me | 4-FPhBF₃K | 90 (98:2) |
| 9 | C(O)Me | 4-FPhLi/CuI | 92 (98:2) |
| 10 | C(O)Me | 4-FPhMgBr/CuI | 96 (98:2) |
| 11 | C(O)Me | MeLi/CuI | 85 (92:8) |
| 12 | C(O)Me | 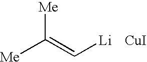 | 80 (88:12) |
| 13 | C(O)Me | 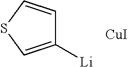 | 84 (94:6) |

Although the Rh-catalyzed conjugate addition methodology was effective for α,β-unsaturated ketone xx, Cu-catalyzed conjugate addition of 4-fluorophenyl magnesium bromide, using trimethylsilyl chloride as an enolate trap, see Varchi, G.; Ricci, A.; Cahiez, G.; Knochel, P. Tetrahedron, 2000, 2727, was more practical providing xx in 94% yield as a 98:2 mixture of epimers after equilibration of the ketone to the thermodynamically favored isomer. The Cu-catalyzed conjugate addition was demonstrated for addition of aryl, vinyl, heteroaromatic, and alkyl metal species, which were unachievable via the Rh-catalyzed conjugate addition methodology.

In conclusion we have reported a method to seamlessly construct highly functionalized cyclopentanoid structures in outstanding selectivity in very few steps. The convergent nature of this approach make it a valuable tool for rapidly assembling structural complexity, and the modular nature of the route allows for the efficient production of structural analogs. The examples described herein are indicative of the diverse array of complex intermediates can be accessed using this chemistry.

We next attempted diastereoselective alkylation of chiral oxazolidinone 3a with halomethylketone derived from the cyclopentane acid 2b.

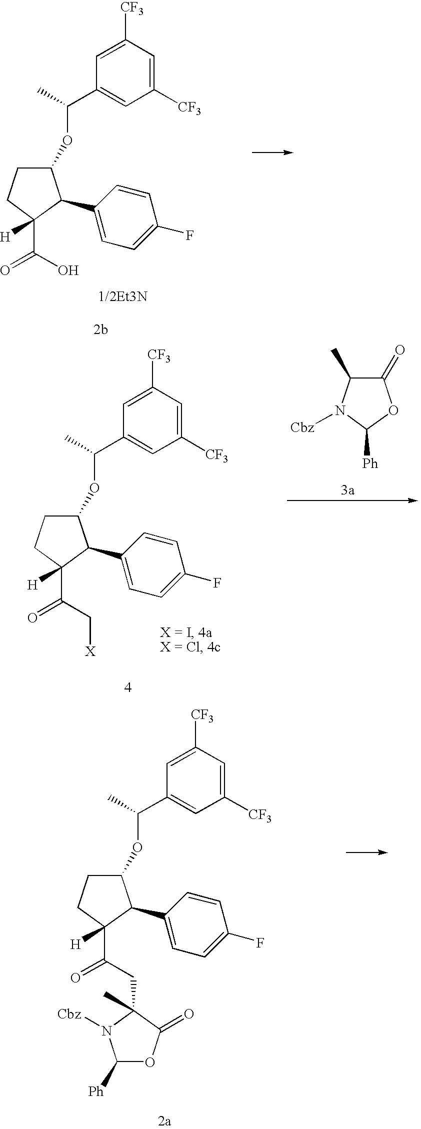

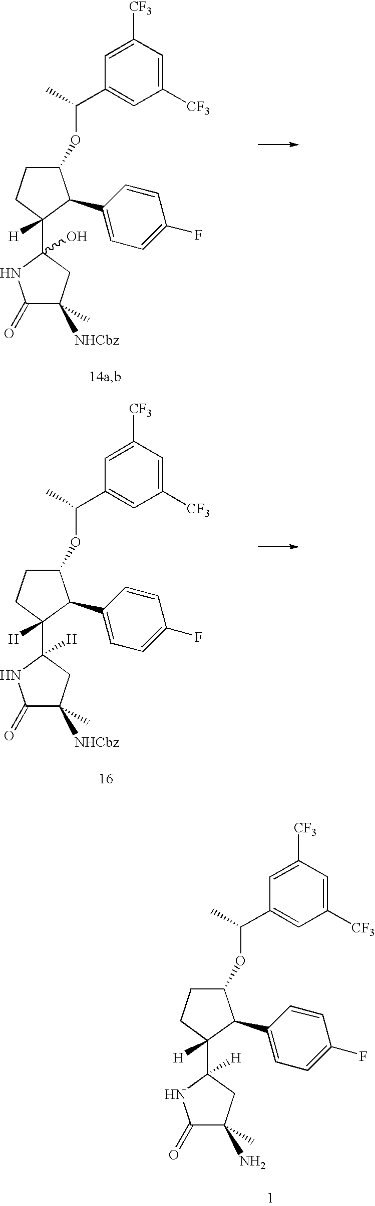

Chloromethylketone 4c was prepared from acid intermediate 2b via 2 steps: i) methyl esterification of 2b which was comprised by treatment of 2b with methyl iodide and an alkali metal carbonate such as Li, Na, K, Ca, or Cs in a polar solvent such as DMF, DMAc, DMSO, or acetone at a temperature range of about 0° C. to about 60° C. Treatment of 2b with MeOH in the presence of acid catalyst such as sulfuric acid, TsOH, MsOH, or benzenesulfonic acid at a temperature range of about 50° C. to reflux may also employ the methylation, ii) chloromethylation of methyl ester 2c which was comprised by treatment of 2c with ClCH₂CO₂H and a metal amide of Formula M⁴N(R₁₆)₂ or M⁴N(Si(R¹⁶)₃)₂, wherein M⁴ is Li, Na, K, or Mg (Mg is divalent, need to be changed), and each R¹⁶ was independently selected from C₁₋₄alkyl in an aprotic organic solvent, for example THF at a temperature range of about −20° C. to about 40° C. ClCH₂I or ClCH₂Br may also employ this reaction.

Compound 4a was prepared by treatment with an alkali metal or ammonium iodide such as LiI, NaI, KI, R₄NI, wherein R is selected from H or C₁₋₄alkyl, in a polar organic solvent such as DMF, DMAc, DMSO, or acetone at a temperature range of about 0° C. to about 60° C. Anhydrous conditions gave a better yield.

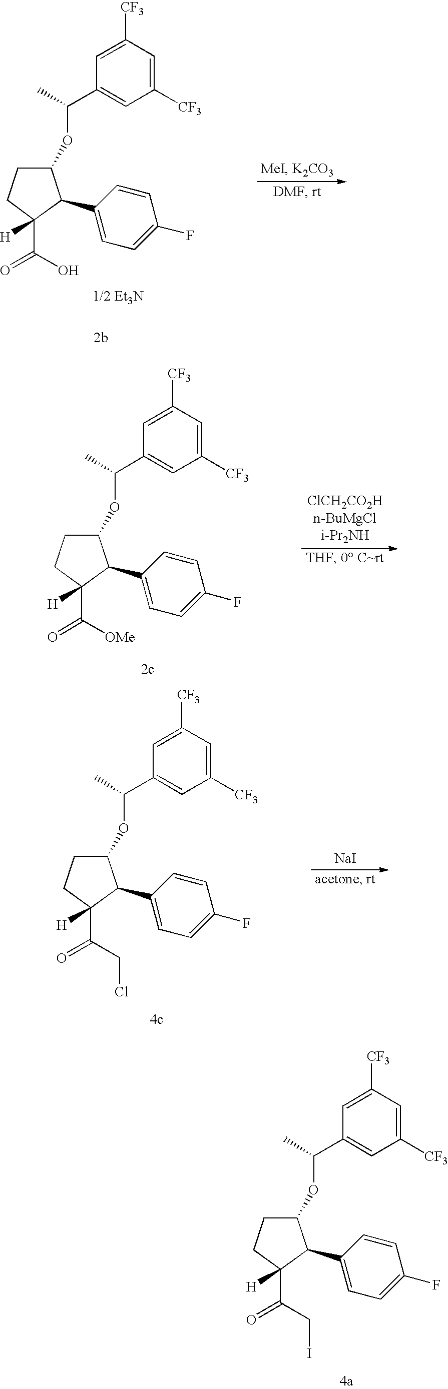

The process for the key intermediate 2a synthesis formed embodiment of this invention. The process comprised a quarterly chiral carbon center by addition of enolate of 3a to α-haloketone 4, under the conditions of an alkali metal amide of Formula MN(R)₂ or MN(SiR₃)₂, wherein M was Li, Na, or K, and each R was independently selected from C₁₋₄alkyl with or without an aprotic polar solvent or amine additive such as DMPU, DMI, or TMEDA in an organic solvent such as toluene or THF at a temperature range of about −78° C. to about −40° C. The combination of less polar solvent and an aprotic polar solvent at lower reaction temperature gave better yield.

The residual excess amount of 3a in the solution of 2a in an organic solvent such as toluene was able to be removed by selective hydrolysis with aqueous LiOH followed by aqueous NaHSO₃ treatment at a temperature range of about 0° C. to about 40° C.

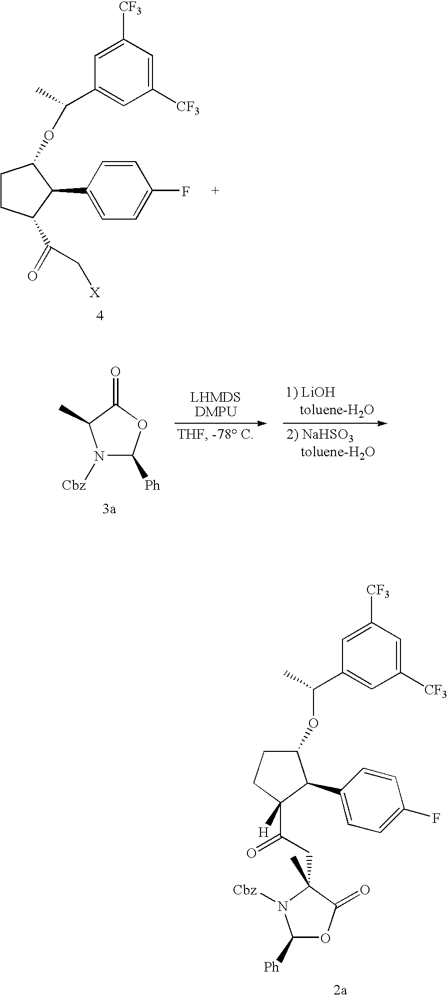

Ammonolysis of the alkylated oxazolidinone 2a with an aqueous or organic NH₃ in an organic solvent such as THF, DME, methanol, ethanol or isopropanol at a temperature range of about 0° C. to about 60° C. directly gave a diastereomer mixture of animals 14a,b.

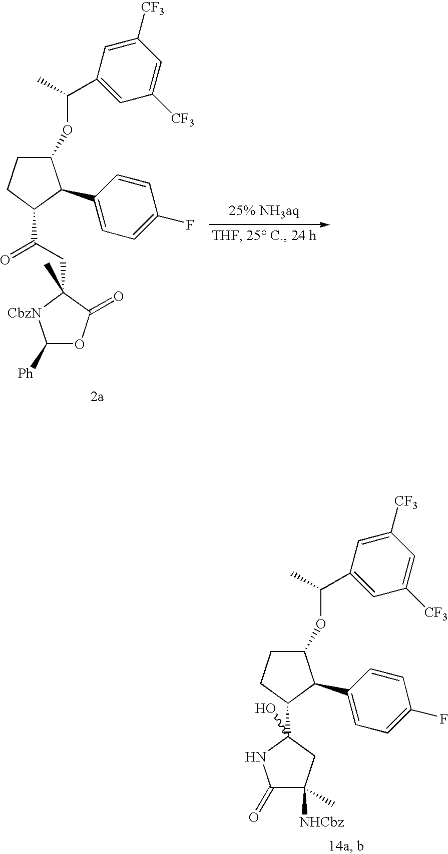

Hydroxyl group in 14a,b was reduced to yield 16 by treatment with a silane of Formula R₃SiH, wherein R was selected from H or C₁₋₄alkyl, in the presence of an acid such as trifluoroacetic acid, methanesulfonic acid, trifluoroborane etherate, or the like in organic solvent such as acetonitrile, toluene, acetic acid, nitromethane, or nitroethane, or neat, at a temperature from about −40° C. to about 60° C., until the reaction was complete, usually about 2 to 24 hours. It was found that this reduction proceeded through enamine intermediate 15a,b, which was converted from 14a,b by treatment with an acid such as trifluoroacetic acid, methanesulfonic acid, trifluoroborane etherate, or the like in organic solvent such as acetonitrile, toluene, ethyl acetate, acetic acid, nitromethane, or nitroethane, or neat, at a temperature from about −40° C. to about 60° C. Compound 16 was produced from both 14a,b and 15a,b under the same conditions and obtained concomitant with its diastereomer 16a. Better yield and diastereoselectivity were obtained by the reduction from enamine 15a,b in acetonitrile at lower temperature.

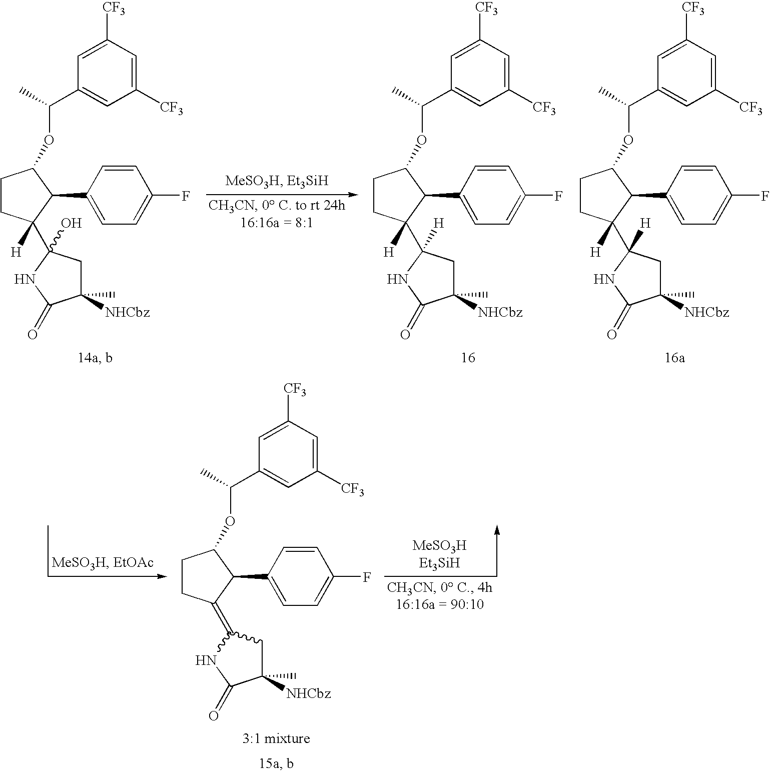

The benzyloxycarbonyl (Cbz) group of 16 was cleaved to give 1 by a noble transition metal, such as Pd/C, Ph(OH)₂/C, Pt/C, catalyzed hydrogenation in an alcoholic solvent such as methanol, ethanol, or the like, or by acidic solvolysis with a hydrogen halide such as HCl, HBr, or HI or an equivalent such as combination of a metal or an ammonium halide and an acid trifluoroacetic acid, methanesulfonic acid, or the like in organic solvent such as acetonitrile, toluene, or acetic acid at a temperature from about 0° C. to about 60° C. If this deprotection of Cbz group was carried out under acidic condition, 1 could be obtained from 14a,b or 15a,b in a one-pot manner. Benzyl halide, which was a side product of deprotection of Cbz group, was easily removed from the reaction mixture by back-extraction with heptane. Free base of 1 was difficult to be extracted from the aqueous layer to organic layer because of its amphipathic physical property. A mixed solvent of t-BuOH and MTBE was found to be effective to extract the free base from the aqueous layer. Crude free base was purified by crystallization as a benzenesulfonate salt from IPA-IPAc-heptane solvent system. The benzenesulfonate salt has two crystal forms Form I or II. Form II would be more thermodynamically stable than Form 1. The benzenesulfonate salt can be recrystallized from acetonitrile and alcohol such as methanol, ethanol, or isopropanol, if necessary.

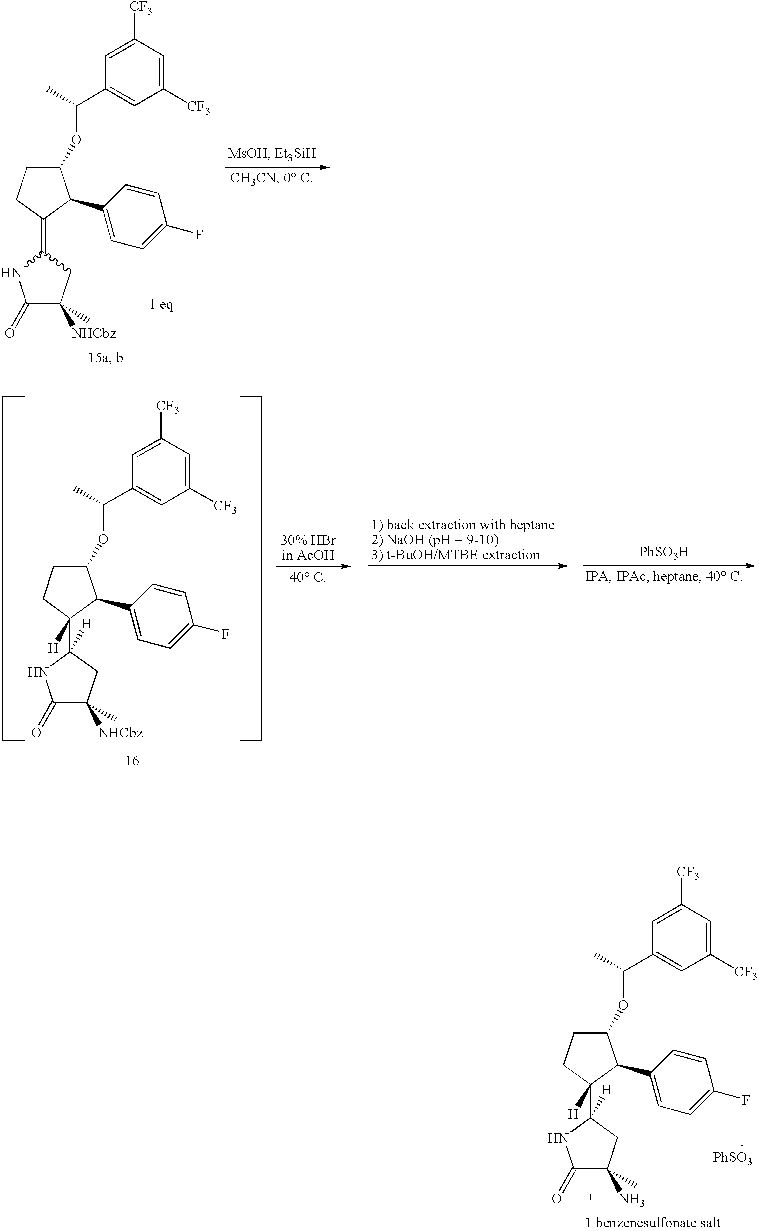

A highly convergent, efficient, and completely diastereo- and enantioselective synthesis of the cyclopentane core of the lactam hNK-1 receptor antagonist (1) has been identified. The general methodology described herein was applied to the synthesis of both the acid intermediate (2) and the iodoketone intermediate (3), which have both been demonstrated to deliver 1 in good yield and acceptable purity.

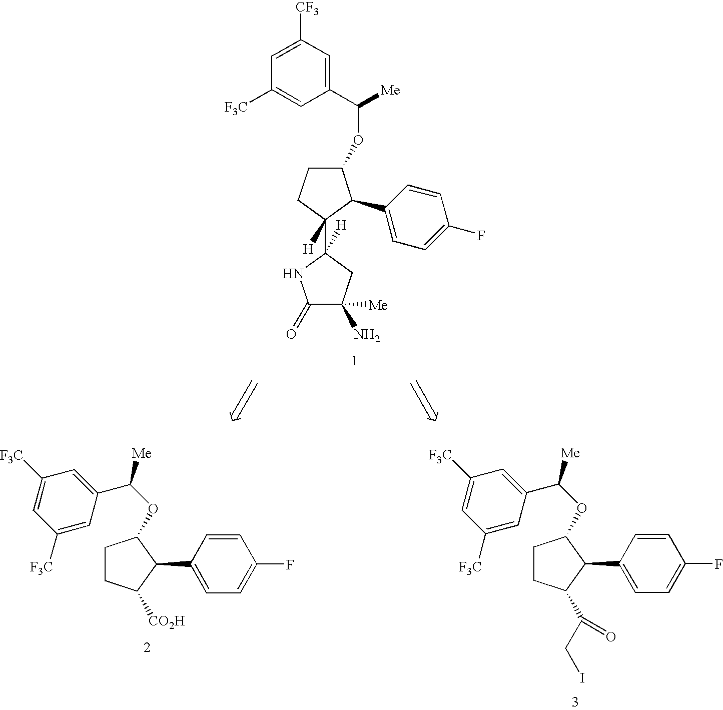

Early development work for making 1 had focused on an efficient synthesis of 2, primarily because it was the only intermediate that had been successfully converted to 1. We successfully developed new methodology to rapidly produce 2 in a highly convergent, diastereo- and enantioselective manner (Scheme 1). The highlights of the synthesis are a highly enantioselective enzymatic reduction of 3-carboxymethyl cyclopentenone (5), a stereospecific Pd-catalyzed etherification, and a diastereoselective Rh-catalyzed conjugate addition.

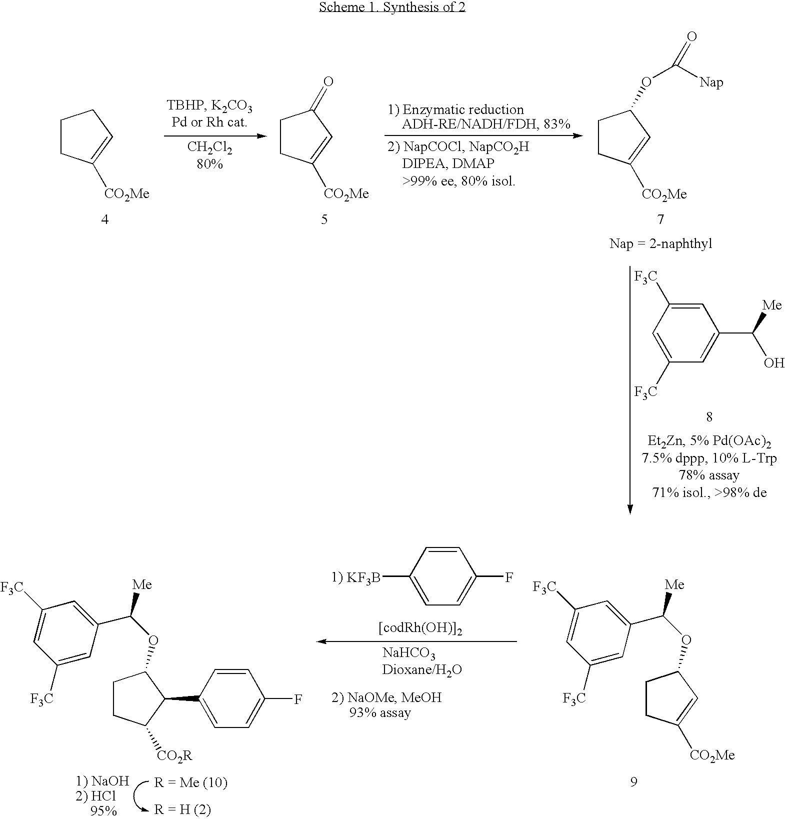

Although the synthesis was an improvement over the existing method to produce 2, the route suffered from difficulties in preparing 5 on scale and costly Rh-catalyzed conjugate addition methodology. Improvements in the endgame chemistry, demonstrated that 2 could be converted to 1 in 7 steps (Scheme 2). An examination of this improved route revealed 3 as a better synthetic target for a convergent synthesis to 1, because several steps were required to convert 2 to 3.

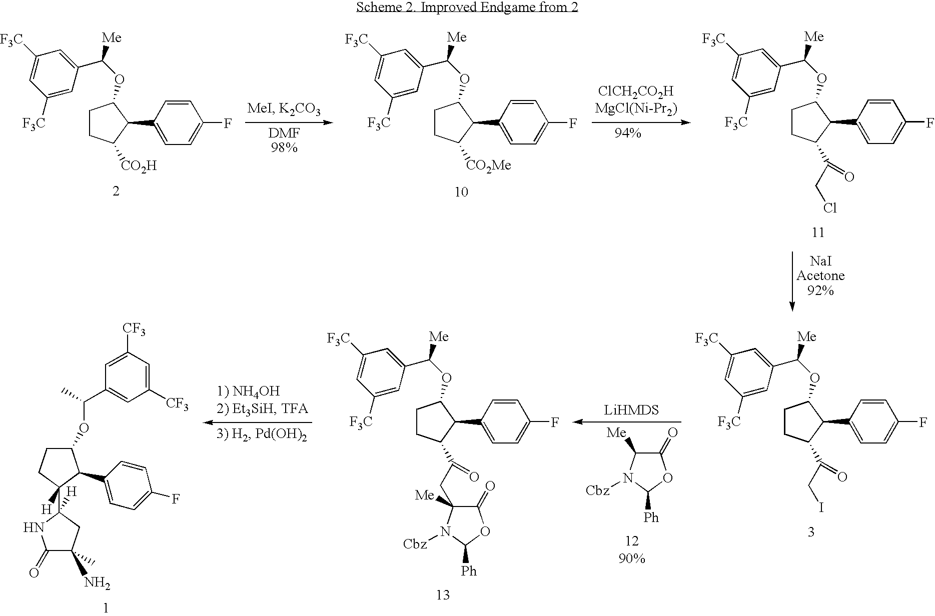

The general methodology developed for the synthesis of 2 was modified to accommodate the changes in substrate necessary to produce 3 (Scheme 3). By directly targeting 3, the overall process was improved by providing an accessible method to produce large quantities of the starting material, transforming the Rh-catalyzed conjugate addition to a Cu-mediated conjugate addition, and eliminating several steps the process. The optimized synthesis of 1 was reduced to 10 steps from the cyanoketone starting material (4 steps from the iodoketone). The discovery and demonstration of this methodology to both 2 and 3 on 30 g scale is described below.

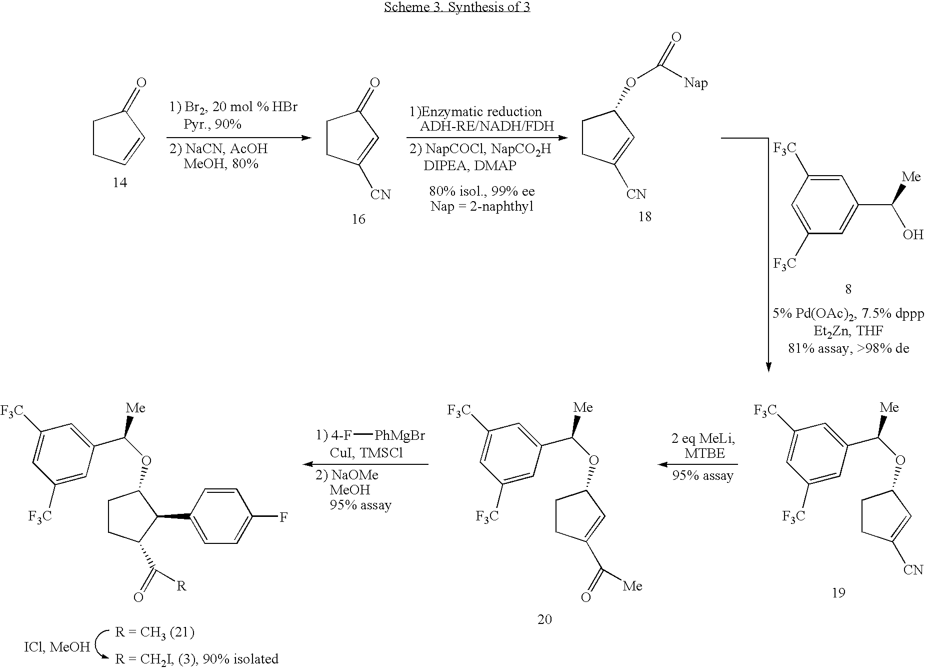

TABLE 2
Pd-catalyzed Etherification of Allylic Esters

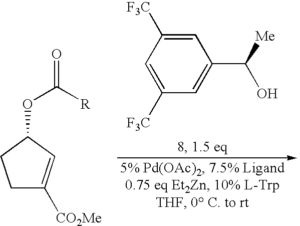

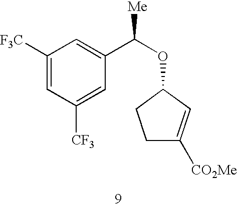

| Entry | R | Assay yield |
|---|---|---|
| 1 | CF₃ | 22 |
| 2 | OtBu | 59 |
| 3 | Me | 60 |
| 4 | p-NO₂Ph | 73 |
| 5 | Ph | 78 |
| 6 | 2-Nap | 78 |

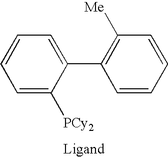

A rudimentary solvent screen did not reveal any solvents better than THF (Table 3), which was used in the original etherification conditions. It is still possible that other coordinating ether-type solvents (or possibly tertiary amine additives) could provide an improvement. This has not been investigated yet.

TABLE 3
Pd-catalyzed Etherification Solvent Screen

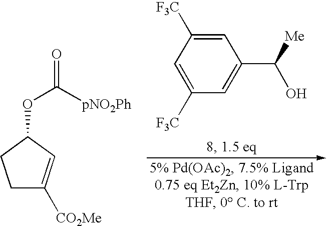

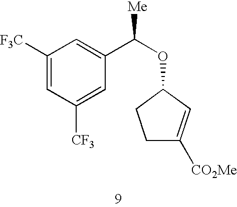

| Solvent | Assay yield |
|---|---|
| t-AmOH | 2.3 |
| DMAc | 30 |
| THF | 73 |
| CH₂Cl₂ | 55 |
| PhMe | 54 |

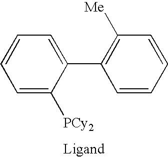

An extensive screening of ligands (Table 4) revealed that the dicyclohexylphosphino variants of the Buchwald biarylphosphine ligands performed far better than the corresponding di-tert-butylphosphino versions. This indicated that relatively unhindered phosphines were preferred in the Pd-catalyzed etherification. In general, the Buchwald biarylphosphine ligands provided a slightly higher yield and a faster reaction (usually, 2 h at 0° C.) than chelating diphosphine such as dppp (typically, 2-24 h at rt). Nevertheless, the dppp ligand had the advantage of lower cost and better availability. Regarding the chelating diphosphine ligands, dppp was more effective than either dppe or dppb. Decreasing the catalyst loading to 2% Pd and 3% ligand (using the Buchwald biarylphosphine ligand) resulted in a slightly slower etherification of the p-nitrobenzoate ester although the assay yield was virtually unchanged (96% conv, 70% yield after 7 h at 0° C.).

TABLE 4
Pd-catalyzed Etherification Ligand/Substrate Screen

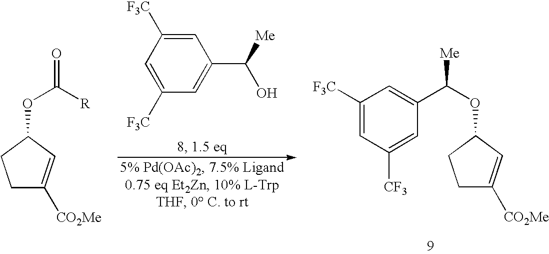

| R = 2-Nap | Ligand: | 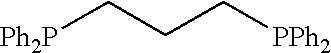 | 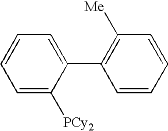 | 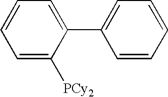 |
|---|---|---|---|---|
| | Assay Yield: | 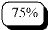 | 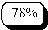 | 73% |

| R = p-NO₂Ph | Ligand: | 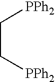 | 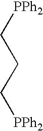 | 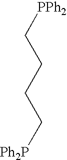 | 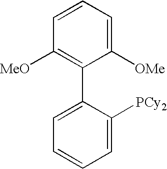 | 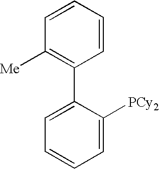 | | 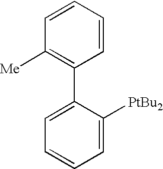 |
|---|---|---|---|---|---|---|---|---|
| | Conditions: | rt, 20 h | rt, 20 h | rt, 20 h | rt, 20 h | rt, 2 h | 0° C., 4 h | rt, 20 h |
| | Conversion | 99% | 99% | 97% | >99% | >99% | 99% | 47% |
| | of Allyl Ester: | | | | | | | |
| | Assay Yield: | 30% | 68% | 38% | 64% | 65% | 73% | 5% |

As a further improvement, it was found that the amount of benzyl alcohol 8 could be reduced to 1.2 equiv in the case of the naphthoate substrate (R=2-Nap) without significantly affecting the assay yield of 9. The combination of the naphthoate ester and dppp ligand provided the best reaction in terms of overall yield and cost-efficiency, and was chosen for further development.

The effect of the reaction concentration was also briefly investigated. A small difference (78% vs 75% assay yield) was observed between the 0.8M and 0.3M reactions in THF (M defined here as mmol of ester substrate vs. mL of the solvent, including the heptane solvent supplied by the Et₂Zn solution). However, the somewhat more productive 0.8M reaction was plagued by formation of a thick oil that was difficult to stir. As a compromise, 0.5M concentration was chosen for the finalized procedure.

Having identified naphthoate ester 7 as the ideal substrate for the etherification, a process was developed for its formation. It should be noted that the acylation of allylic alcohol 6 was a sensitive reaction, and treatment with naphthoyl chloride afforded low assay yields of the desired allylic ester. In contrast, in situ formation of naphthoic anhydride (from acid chloride and acid) followed by treatment with the alcohol provided allylic ester 7 in good assay yield (91%). Isolation by crystallization provided 7 in 77% yield and >99% LCAP.

A 3 g front run for the etherification reaction was carried out using 1.2 eq of alcohol 8, 3% Pd and 4% dppp ligand. Although a 78% assay yield and 75% isolated yield (95% LCAP, 98% assay) was obtained, the reaction was relatively sluggish and took 2 days to >99% completion. Therefore, 4% Pd and 6% ligand were used for the 20 g demonstration.

The reaction on the 20 g scale exhibited a 1 h induction period although no significant exotherm was observed when the reaction started to accelerate. Allylic ether 9 was isolated after a Darco KB-B treatment (to remove Pd), solvent switched to MeOH, and crystallized from MeOH-water. Although a somewhat lower isolated yield was observed on 20 g scale due to increased mother liquor losses (6%), a significantly more pure product was obtained compared to the 3 g front run. In the end, 20 g of 9 was isolated by crystallization in a 71% yield and >98% purity (Scheme 4).

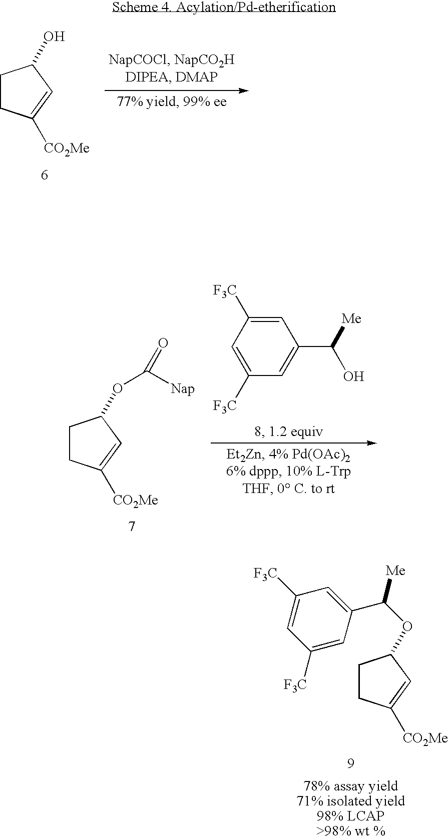

### Diastereoselective Conjugate Addition:

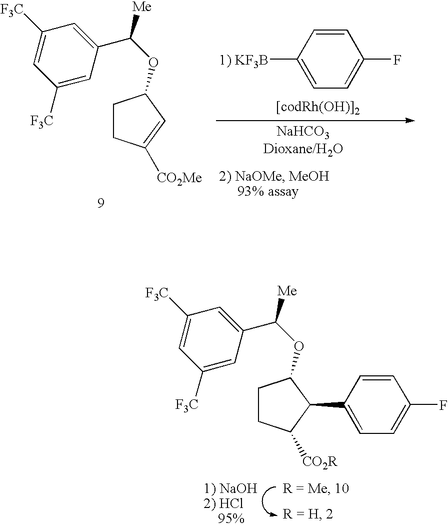

In order to complete the synthesis of the cyclopentane core, a diastereoselective conjugate addition was required. We envisioned that that bulky ether substituent would effectively control the facial selectivity of the incoming nucleophile, affording the desired trans configuration in the conjugate addition product. Isomerization of the ester under thermodynamic conditions should strongly favor the trans configuration, affording trans, trans-1,2,3 trisubstituted cyclopentane 10 in a completely diastereoselective fashion.

The optimal conjugate addition to substrate 9 would involve the addition of 4-fluorophenyl Grignard reagent to the substrate. A literature survey of similar transformations revealed mixed results, affording moderate to low yields (20-60%) of the desired conjugate addition product. Indeed, when the conjugate addition was performed on 9 with 4-fluorophenylmagnesium bromide in the presence of CuI and TMSCl, approximately 45% of the was observed (eq. 7). Further analysis revealed that the conjugate addition product had undergone further 1,2-addition with Grignard to afford diaryl carbinol 27, which was obtained in 40% yield. Attempts to circumvent this overreaction with additives, solvents, or temperature were unsuccessful. In fact, even if the reaction was performed with an undercharge of Grignard reagent, an equimolar ratio of 10 and 27 were observed.

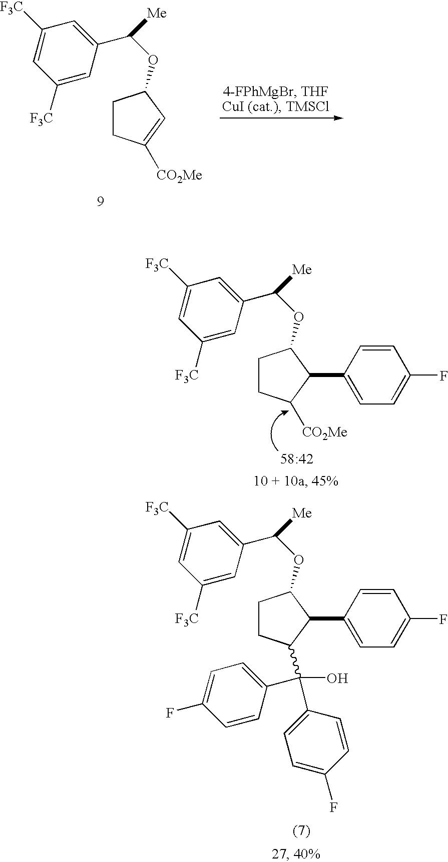

An alternative to Grignard conjugate addition was the Rh-catalyzed conjugate addition with aryl boronic acids. Most of the literature reports in this area involve the use of a chiral ligand to afford asymmetric induction in the conjugate addition product. One of the issues associated with developing the asymmetric variant of this reaction is the facility with which the “ligandless” Rh-catalyzed conjugate addition was accomplished. We suspected that a diastereoselective, Rh-catalyzed conjugate addition should be readily accomplished in the absence of ligand. Indeed, when 9 was subjected to 4-fluorophenyl boronic acid in the presence of [CODRh(OH)]₂ under standard conditions, the desired conjugate addition product (10) was obtained in good selectivity (>99:1). It should be noted that the assay yield of conjugate addition step was strongly influenced by the amount of boronic acid charged to the reaction (Table 5). When only 1.5 equiv of boronic acid was charged, only 32% assay yield was observed. In contrast, when 5 equivalents of boronic acid was charged, 89% assay yield of desired product was observed. This was consistent with literature reports that hydrodeborylation of the boronic acid is a competitive side reaction.

TABLE 5
Rh-catalyzed Conjugate addition

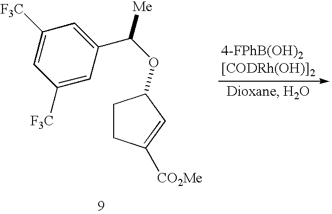

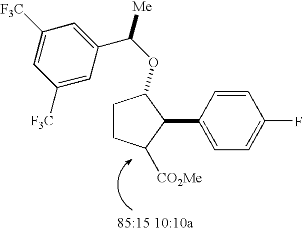

| Equiv Boronic Acid | Assay yield 10+10a | Assay 9 |
|---|---|---|
| 1.5 (25° C.) | 32% | 61% |
| 1.5 (90° C.) | 55% | 37% |
| 3.0 (90° C.) | 76% | 13% |
| 5.0 (90° C.) | 89% | 2% |

Attempts to employ the arylzinc reagent in the Rh-catalyzed conjugate addition were unsuccessful, leading to many impurities and minimal desired product (<10%). In contrast, substituting the aryltrifluoroborate salt was quite successful, allowing the charge of the fluoroborate salt to be reduced to 1.5 equivalents with no loss in assay yield. The reaction was found to have improved performance in EtOH versus that observed in dioxane/H₂O, where 10 was obtained in 93% assay yield using only 1.5 equivalents of the arylfluoroborate salt (eq. 8).

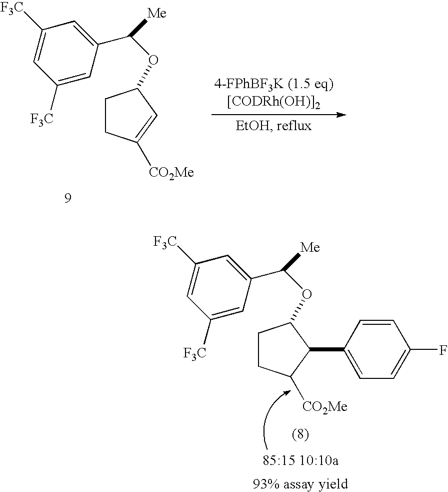

Interestingly, the diastereomeric ratio of 10 to 10a was much higher than that observed in the Grignard conjugate addition (85:15 vs 55:45), presumably due to partial isomerization under the reaction conditions. When the mixture of 10 and 10a were subjected to isomerization conditions (NaOMe, MeOH, 50° C.) the ratio reached equilibrium at 95:5 (Scheme 5). Further heating, or more base did not alter the ratio. The process previously developed required 10 to complete the synthesis of 1; however 10 was converted to 2 in order to demonstrate the feasibility of the isolation of a solid, and to verify the structure and impurity profile by comparison to an authentic sample.

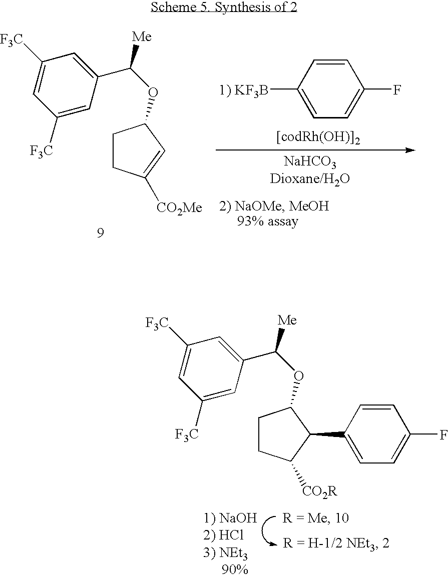

After isomerization, ester 10 could be hydrolyzed in the same pot to acid 2. After neutralization with aqueous HCl, the corresponding acid could be extracted, and isolated as the NEt₃ hemisolvate. The conjugate addition/isomerization process was demonstrated on 20 g, and was hydrolyzed in the same pot to afford 2. 19.7 g of 2 was isolated as the NEt₃ hemisolvate, and the purity of the material was similar to that obtained by the previous method, 98.2 LCAP (1.3 area % epimer derived from 10a).

In summary, the enzymatic reduction/Pd-etherification/Rh-conjugate addition route was demonstrated as an efficient process for the rapid access of the acid intermediate 2. This synthesis is significantly shorter than the original synthesis of this fragment (5 steps vs. >12 steps), avoids the capricious etherification methodology required for the old synthesis, and affords 2 in similar purity and improved overall yield (35% vs <25%).

### Experimental Procedures—Part 1:

### Step 1—Oxidation of Carboxymethylcyclopentene:

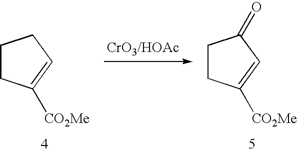

### Procedure:

To a cooled (0° C.) 2 L round bottom flask with a magnetic stir bar and internal temperature probe was added acetic anhydride (615 g, 570 mL, 6.02 mol). Chromium trioxide (214 g, 2.14 mol) was added in portions while maintaining constant stirring and to control the exotherm. The resulting blood red solution was stirred to dissolve the chromium trioxide until the temperature had cooled to 20° C. A 5 L three-neck flask was fitted with an addition funnel, overhead stirring mechanism, nitrogen inlet and internal temperature probe and charged with 4 (100 g, 101 mL, 0.793 mol) in 1.4 L CH₂Cl₂. The oxidizing solution of chromium trioxide and acetic anhydride was charged to the addition funnel and added dropwise to the reaction mixture, maintaining the internal temperature between 10 and 14° C. The initially yellow solution became dark after the first few drops of oxidizer were added.

The reaction was worked up in two equally-sized batches due to limitations on vessel size in the laboratory. Each batch was treated exactly the same way, as follows: The dark, homogeneous solution was poured carefully into a 4 L beaker with an overhead stirring mechanism. The reaction flask was rinsed with 250 mL CH₂Cl₂. 500 mL H₂O was added followed by 10 g NaHCO₃ which resulted in gas evolution. Additional NaHCO₃ (830 g, 10 mol) was added in portions while maintaining 500 rpm stir rate in the viscous mixture. The resulting dark green suspension was diluted with 1 L H₂O and filtered through a 3 L fritted funnel containing a 1 cm pad of solka floc. The biphasic solution was extracted with CH₂Cl₂ (3×1 L) and the combined organics dried using MgSO₄, then filtered and the resulting solution was concentrated in vacuo to afford a pale green oil. Distillation through a 30 cm Vigreux column followed by recrystallization from MTBE:hexane (1:10, 55 mL total) provided 38.4 g of 5 as a white crystalline solid (35%).

### Step 2—Enzymatic Reduction:

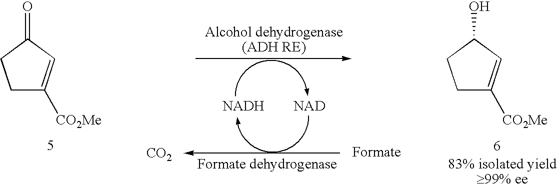

### Procedure:

To potassium dibasic buffer (100 mM, pH 7.0, 2 L), sodium formate (120 g) and nicotinamide adenine dinucleotide (NAD, 8 gram) was added which reduced the buffer pH to 6.7. The enzymes were added to the buffer: alcohol dehydrogenase RE (5 g, 185 KU), formate dehydrogenase (20 g, 94 KU). Substrate 5 (10 g, 0.071 mol) was added directly as a powder and the temperature controlled at 25° C. with the pH controlled at 6.5 using 2N sulphuric acid. Reaction was aged for 24 hours then extracted with ethyl acetate (2 volume extractions) followed by vacuum concentration. Overall yield of 6 was 83% with 2% loss from extraction and <3% residual enone. The remaining 13% mass balance was determined to be enone loss from instability in aqueous.

### Step 3—Acylation of Allylic Alcohol:

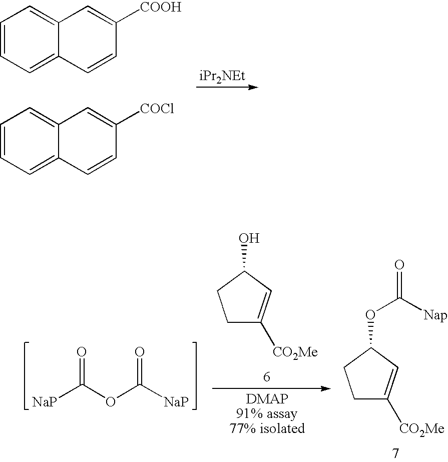

### Procedure:

A suspension of 2-naphthoic acid (24.6 g, 143 mmol) and 2-naphthoyl chloride (27.2 g, 143 mmol) in dichloromethane (200 mL) was cooled to an internal temperature of +5° C. in an ice bath. Diisopropylethylamine (89 mL, 511 mmol) was added while maintaining the internal temperature below 24° C. When the exotherm subsided, the ice bath was replaced with a room temperature water bath. Initially, a brown, clear solution formed, which gradually turned into a fine slurry, which was stirred at room temperature for 30 min. A solution of 6 (14.4 g assay, 102 mmol, >99% ee) and DMAP (1.25 g, 10.2 mmol, 0.10 equiv) in dichloromethane (50 mL) was added in one portion. A very mild exotherm was observed, the temperature reached maximum at 23° C. The reaction mixture was stirred at room temperature for 2.5 h. Water (10 mL) was added and the reaction mixture was stirred for 2.5 h at room temperature. HPLC analysis at this point indicated complete hydrolysis of the excess naphthoic anhydride. The reaction mixture was combined with MTBE (500 mL) and washed with saturated aq NaHCO₃ (2×500 mL), water (500 mL), IM aq HCl (500 mL) and finally water (4×500 mL). The dark brown, cloudy organic phase was filtered through a short pad of Solka Floc to provide 91% assay yield in the filtrate. The solution was concentrated to 200 mL volume, heptane (200 mL) was added, and the mixture was filtered through ˜10 g of silica gel eluting with 100 mL of heptane-MTBE 2:1. The filtrate was concentrated to 200 mL volume and seeded. Another 50 mL of solvent was removed at 40° C., and the remaining suspension was stirred while allowed to cool to room temperature over 2 h. After 15 h at rt, the suspension was filtered and the filter cake was washed with 50 mL of heptane (mother liquor losses: 4%) to give 23.2 g of 7 as a fine white powder in 77% yield and 99% LCAP.

### Step 4—Pd-Catalyzed Etherification Reaction:

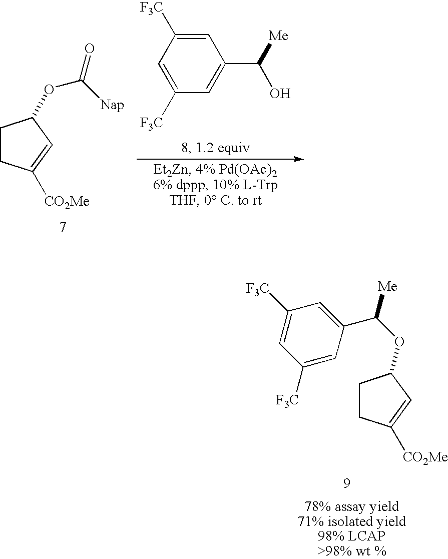

### Procedure:

A 250 mL round bottom flask was charged with 8 (23.3 g, 90.3 mmol, 1.2 equiv), evacuated, and backfilled with nitrogen. THF (75 mL) was added, and the resulting solution was cooled in an ice bath to +5° C. 1.0M Et₂Zn in heptane (45 mL, 45 mmol) was added, which resulted to a moderate exotherm to 15° C. The cooling bath was removed and the resulting clear solution was stirred at room temperature for 1 h.

A separate 500 mL round bottom flask was charged with 7 (22.3 g, 75.3 mmol), Pd(OAc)₂ (676 mg, 3.01 mmol, 4 mol %), 1,3-bis(diphenylphosphino)propane (1.86 g, 4.51 mmol, 6 mol %), and L-tryptophan (1.54 g, 7.54 mmol, 10 mol %). The flask was then evacuated, backfilled with nitrogen, and cooled in an ice bath. The alkoxide solution from the first flask was transferred via cannula into the second flask. The cooling bath was removed, and the resulting green-brown suspension was stirred at room temperature for 16 h. HPLC analysis at this point indicated complete conversion of the naphthoate ester. The light brown suspension was cooled in an ice bath and combined with 1M aq HCl (100 mL) and MTBE (100 mL), which resulted in an exotherm to 15° C. The suspension was stirred at 5° C. for 15 min and then filtered through Solka Floc eluting with MTBE (200 mL). The light yellow filtrate was washed with water (3×200 mL), 5% aq NaHCO₃ (2×300 mL), and water (2×200 mL). HPLC analysis of the organic phase revealed 78% assay yield of the product. Darco KB-B (15 g) was added to the organic phase, and the suspension was stirred at room temperature for 3 h, then filtered through Solka Floc. The nearly colorless filtrate was solvent switched to MeOH with the final volume of 120 mL. Water (5 mL) was added, the solution was seeded, and additional water (70 mL) was added dropwise over 15 min. The slurry was stirred at room temperature for 1 h and then filtered. The resulting white crystals were dried under vacuum to provide 20.4 g of 9 in 71% yield and 98% LCAP.

### Step 5—Rh-Catalyzed Conjugate Addition/Hydrolysis/Acid Isolation:

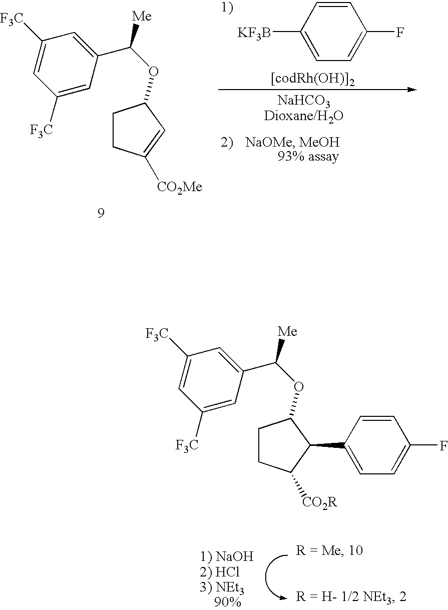

### Procedure:

To a 250 mL, 3-necked round bottom flask equipped with magnetic stirrer, thermocouple, and nitrogen inlet was added 9 (19.0 g, 0.0497 mol), NaHCO₃ (1.94 g, 0.023 mol), fluoroborate salt (16.1 g, 0.080 mol), and [CODRh(OH)]₂ (646 mg, 0.0015 mol). The flask was sealed and purged with nitrogen for 1 hour. Ethanol (150 mL) which had been degassed with nitrogen for 1 hour was charged to the reaction vessel containing the solids via cannula, and the reaction mixture was heated to 90° C. The reaction was complete by HPLC analysis within 3 hours. The mixture was cooled to 50° C., and charged with 11 mL of 25 wt % NaOMe in MeOH. The mixture was aged at 50° C. for 4 hours which showed a diastereomer ratio of 95:5 upon complete equilibration. The mixture was cooled to 40° C., charged with 3N KOH (40 mL), and aged at 40 C for 1 h which showed complete hydrolysis by HPLC analysis. The mixture was cooled to room temperature, diluted with 270 mL water and 270 mL heptane. The heptane layer was removed, and the aqueous layer was mixed with 300 mL heptane, and neutralized with 50 mL of concentrated HCl. The layers were separated and the heptane layer was washed twice with water (200 mL). Assay of the heptane solution showed 18.4 g of 10 (83%). The heptane solution was azeotroped to a Kf<500, then concentrated to a volume of 150 mL. The solution was diluted with MTBE (15 mL), warmed to 45° C., and NEt₃ (3.0 mL, 0.0215 mol) was added. After the batch was aged for 15 min, the mixture was seeded (10 mg) and the batch was slowly cooled to room temperature over 2 hours. The batch was aged for 1 hour, assayed the supernatant (10.8 mg/mL), and filtered over a sintered glass funnel. The resulting crystalline solid was washed with 50 mL of 10:1 heptane:MTBE, affording 19.4 g of 2 (76%). Loss to mother liquors=1.95 g (7.6%). Analysis of the isolated solid showed a purity of 98.0 wt %, 98.2 LCAP, the largest impurity was the diastereomer derived from 10a (1.3 area %). The benzylic diastereomer was also present at 0.3 area %.

### Step 6—Methyl Esterification/Chloromethylation/Iodination:

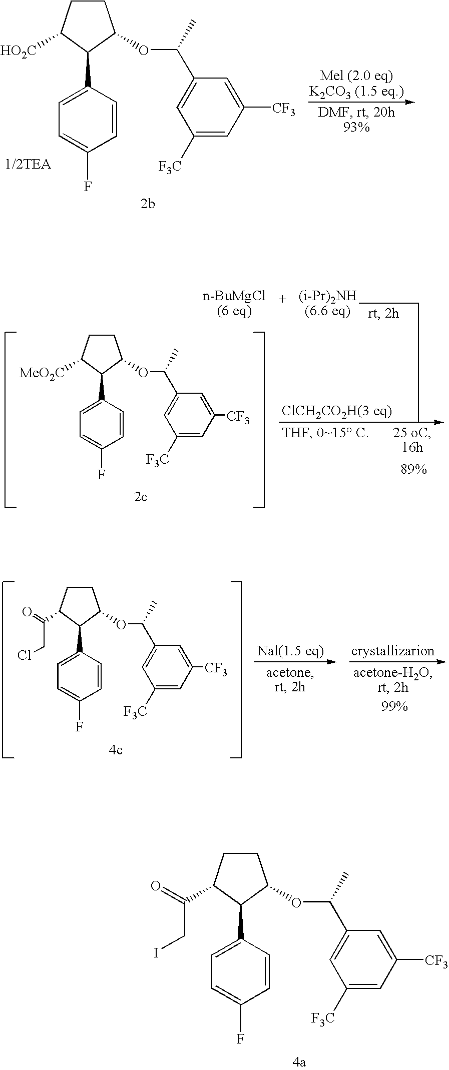

### Procedure:

Methyl iodide (38.2 mL, 613 mmol) was added to a suspension of K₂CO₃ (63.5 g, 460 mmol) and 2b (160 g, 98.6 w %, 306 mmol) in DMF (480 mL) at 23° C. over 30 min. The mixture was stirred at room temperature for 20 h. To the reaction mixture were added MTBE (1280 mL), 10% brine (800 ml), and H₂O (800 ml). The organic layer was separated and washed with 0.5N phosphate buffer (800 ml, pH 6.8) and 10% brine (800 ml). The organic layer (1240 ml) was azeotropically dried with MTBE at 40° C. and then solvent was switched to THF at 40° C. This crude 2c in THF solution (480 ml, 136 g assay, 283 mmol, 92.5%, KF<400 ppm) was used for the next step without further purification.

To a solution of 1.9 M n-BuMgCl in THF (6 eq., 1700 mmol, 895 mL) was added diisopropylamine (6.6 eq., 1870 mmol, 262 mL) at 25˜30° C. and the resulting slurry was stirred for 2 h at 20˜25° C. A mixture of 2c (136 g, 238 mmol) and chloroacetic acid (3 eq., 850 mmol, 80.3 g) in THF (678 mL) was dropwise added to the slurry over 1 h below 15° C. The reaction mixture was stirred for 16 h at 20-25° C. and then transferred to a mixture of 6 N HCl aq. (15 eq, 4.25 mol, 709 mL) and MTBE (1360 mL) over 30 min below 15° C. The organic layer was separated and washed with 0.5N phosphate buffer (800 ml, pH 6.8) until the pH of the aqueous layer was >6.0 and then with 23% brine. The organic layer was azeotropically dried with MTBE until KF of the solution <500 ppm and then adjusted the total volume to 1440 mL. Assay yield of 4c by HPLC was 125 g (251 mmol, 88.7%).

A solution of 4c (125 g, 251 mmol) in MTBE (1440 mL) was solvent-switched to acetone and the total volume was adjusted to 1120 mL. To the solution was added NaI (56.5 g, 377 mmol, 1.5 eq.) and acetone (125 ml) were added at ambient temperature. The mixture was stirred for 16 h at ambient temperature. The reaction mixture was dropwise added to a suspension of seed crystal of 4a (125 mg) in water (2120 mL) over 1 h and the reaction vessule was rinsed with acetone (125 ml). After 2 h aging, the crystals of 4a were collected by filtration and washed with water until the filtrate became pH>4.5. After drying, 4a was obtained as yellow crystals (153 g, 93.8 wt %, 99.2% yield).

### Step 7—Oxazolidinone Coupling:

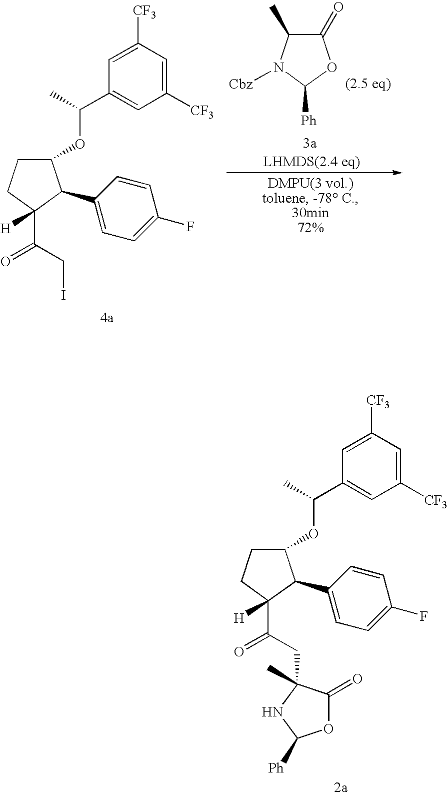

### Procedure:

To a mixture of DMPU (15.0 mL) and DMI (15.0 mL) in toluene (100 mL) was added 1.0 M LHMDS in hexane (40.8 mL, 40.8 mmol, 2.4 eq.) at 20˜25° C. The resulting mixture was stirred for over 5 min, then cooled below −75° C. A solution of oxazolidinone 3a (13.2 g, 42.5 mmol, 2.5 eq.) in toluene (100 mL) was slowly added to the solution below −70° C. over 1 h and the resulting solution was stirred for 30 min below −70° C. Then a solution of 4a (10.0 g, 17.0 mmol) in toluene (50.0 mL) was slowly added to the above solution below −70° C. over 1 h. After stirred for 30 min below −70° C., the reaction mixture was quenched by 10% aqueous citric acid q (100 mL) and then warmed to ambient temperature. The organic layer was separated, washed with 10% NaHCO₃ aq (50 mL) and then treated with 1N LiOH aq. (100 mL, 100 mmol, 5.9 eq.) for 18 h at ambient temperature. After the aqueous layer was separated, the organic layer was washed with 1N NaHSO₃ (100 mL, 100 mmol, 5.9 eq.) and 10% brine (50.0 mL). The product 2a was obtained as a toluene solution (9.44 g, 72%, 254 mL) and used for the next reaction without farther purification.

### Step 8—Enamine Formation:

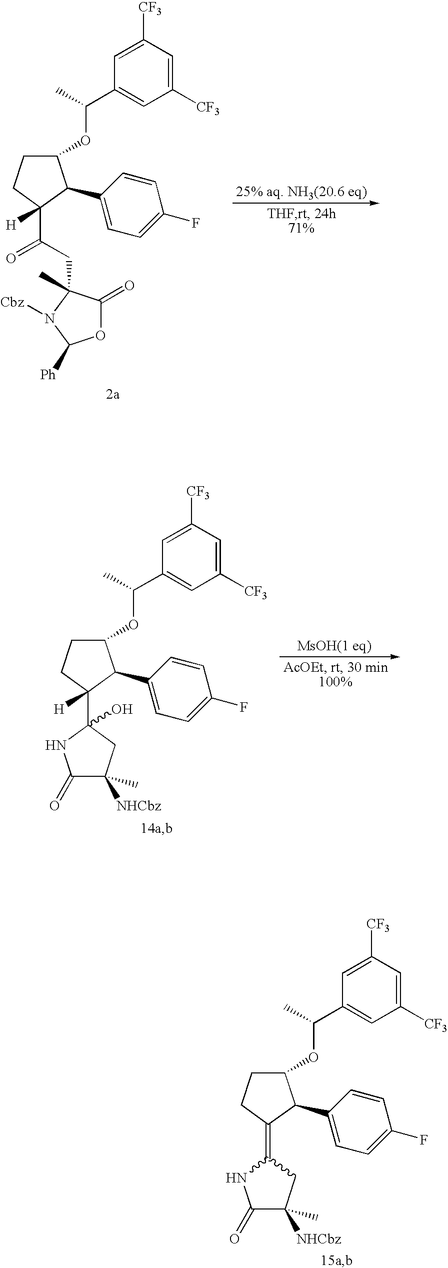

### Procedure:

Toluene solution of 2a (2.02 gA, 2.62 mmol) was solvent-switched to THF (ca. 23.0 mL) at 40° C. and then treated with 25% aqueous NH₃ (20.6 eq, 4.04 mL, 54.0 mmol) at ambient temperature for 24 h. To the reaction mixture were added 5% brine (10.1 mL) and AcOEt (10.1 mL) The organic layer was separated and washed with 10% aqueous NaHSO₃ (10.1 mL×2), 10% aqueous K₂HPO₄ (10.1 mL), and 20% brine (10.1 mL). Animal 14a,b was obtained as an AcOEt solution (1.23 g, 1.80 mmol, 70.8%, 31.8 mL).

The AcOEt solution of 14a,b (4.04 g, KF 4.6%) was azeotropically dried with AcOEt and concentrated to 20.2 mL (KF<0.3%) at 40° C. Then, MsOH (0.31 mL, 1 eq) was added to the mixture at 0˜5° C. and the reaction mixture was stirred for 1 h at 0˜5° C. Water (20.2 mL) was added to the reaction mixture and the organic layer was separated, washed with 10% aqueous K₂HPO₄ (20.2 mL) at 0˜10° C., with 20% aqueous NaCl (20.2 mL) at 15˜25° C. in turns, and treated with active charcoal (Shirasagi P: 312 mg and Darco KB-B: 312 mg) at 24˜27° C. for 30 min. The mixture was filtered and rinsed with AcOEt (12.1 mL). The filtrate and washing were combined and solvent-switched to CH₃CN, and concentrated to 15.9 mL. Enamine 15a,b in CH₃CN was obtained as a mixture of regioisomers (3.19 g, 100%, isomers ratio 76:24).

### Step 9—Endgame (Silane Reduction/Deprotection/Crystallization):

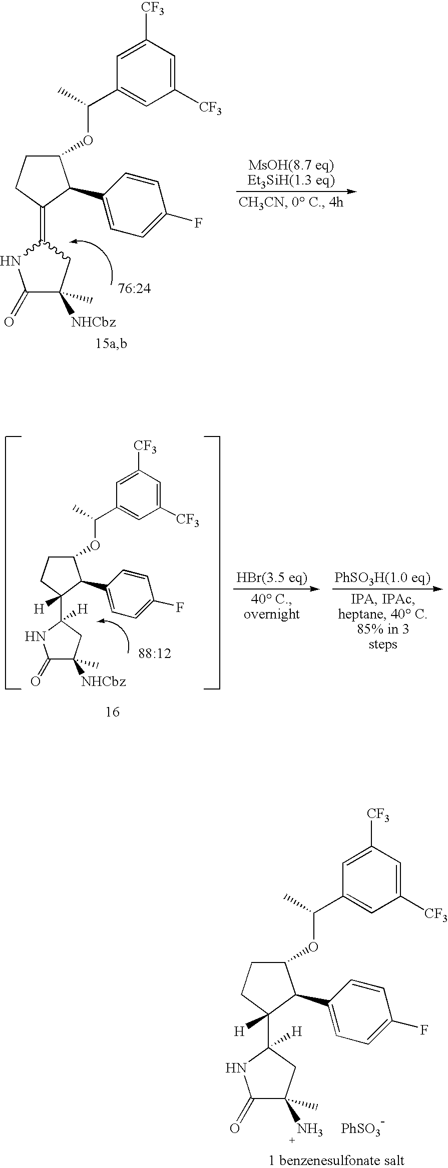

### Procedure:

MsOH (5.85 mL, 80.3 mmol, 8.7 eq.) and Et₃SiH (1.93 mL, 12.1 mmol, 1.3 eq.) were successively added to a solution of enamine 15a,b in CH₃CN (30 mL, 6.15 g, 9.25 mmol, 1 eq.) below 0° C., and then the resulting mixture was stirred for 4 h at −5˜0° C. After complete consumption of 15a,b, 30% HBr in AcOH (3.20 mL, 16.1 mmol, 3.5 eq.) was dropwisely added below 5° C. The mixture was warmed to 38-42° C., stirred overnight at the same temperature, and then cooled to 0° C. To the reaction mixture were successively added water (30.8 mL) and active charcoal (Shirasagi P, 1.23 g). The resulting mixture was stirred for 1 h then filtered, and rinsed with CH₃CN/H₂O=1/1 (18.5 mL). The filtrate and washings were combined and washed with heptane (61.5 mL×3). The aqueous solution was adjusted to pH 3˜4 with 5N NaOH_{aq} (17 mL) below 20° C. MTBE/t-BuOH=2/1 (30.8 mL) was added to the reaction mixture and the resulting mixture was basified with 5N NaOH_{aq} (19.5 mL) below 20° C. to pH=9˜10. After the organic layer was separated, the aqueous layer was extracted with MTBE/t-BuOH=2/1 (30.8 mL). The vessels were rinsed with MTBE/t-BuOH=2/1 (12.3 mL). The organic layers were combined and washed with 12% pH=6.5 phosphate buffer (30.8 mL) and 23% brine (30.8 mL) in turns. The organic solution was solvent-switched to IPAc (KF=444 ppm) to become heterogenous. The IPAc suspension was filtered and the filtered solid was washed with IPAc (12.3 mL). The combined filtrates was concentrated to 35 mL. Compound 1 in IPAc was obtained as a brown solution (4.93 g, 100%) as a free base.

A solution of 1 free base in IPAc (221 mg/mL, 5.0 mL, 1.11 g, 2.08 mmol, 1 eq.) was diluted with IPAc (9.43 mL). To the solution was dropwisely added PhSO₃H.H₂O in IPA (1.5 M, 1.38 mL, 2.08 mmol, 1.0 eq.) over 2 h at 40° C. The resulting slurry was stirred overnight at 40° C. and heptane (16.7 mL) was added to the slurry over 1 h. After stirred for 21 h at 40° C., the slurry was cooled to ambient temperature. The product was collected by filtration, washed with IPAc/heptane=1/1 (8.3 mL×2), and dried in vacuo at 40° C. overnight. Compound 1 was obtained as a colorless solid (1.21 g, 99 wt %, 84.6%, Form I) as a benzenesulfonate salt.

### Experimental Procedures—Part 2:

### Step 1—Bromination of Cyclopentenone:

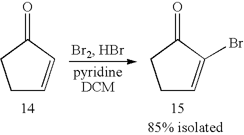

### Procedure:

A 3 L round bottom flask with a magnetic stir bar, nitrogen inlet, addition funnel and internal temperature probe was charged with 2-cyclopentenone (100 g, 1225 mmol) in 1.25 L CH₂Cl₂ and cooled to −20° C. HBr (27.3 mL, 245 mmol) was added and the light yellow solution stirred for 5 min. The addition funnel was charged with bromine (61.9 mL, 1225 mmol) and it was added dropwise over 1 h while maintaining the internal temperature between −24 and −20° C. The bromine was decolorized rapidly during the addition. The yellow solution was stirred at −20° C. for 30 min until TLC indicated consumption of starting enone. Pyridine (149 mL, 1837 mmol) was added dropwise, maintaining the internal temperature below −20° C. Upon complete addition, the solution was stirred at 0° C. for 1 h. The reaction was quenched with 1 M Na₂S₂O₃ (1 L) and diluted with MTBE (2 L). The organic phase was washed with 1 M HCl (2×1 L) followed by H₂O (1 L). The dark organic phase was dried using Na₂SO₄ then filtered. The remainder of the CH₂Cl₂ was solvent switched to MTBE until an ultimate ratio of MTBE:CH₂Cl₂ of 8:1 was reached. The dark solution was stirred with 30% (60 g) DARCO KB-B overnight. The DARCO was removed by filtration through a short pad of solka floc to afford a colorless solution. Solvent was removed in vacuo to provide 170 g of 15 as a white crystalline solid (95.83 wt %, 87% isolated yield).

### Step 2—Cyanation/Elimination:

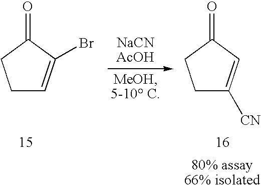

### Procedure:

To a solution of 15 (62.5 g assay; 0.388 mol) and AcOH (22 mL, 0.384 mol) in MeOH (400 mL) at 15° C. (due to endothermic dissolution) was added solid NaCN (CAUTION: highly toxic; 28.5 g, 0.582 mol, 1.5 equiv). The temperature rose from 15° C. to 30° C. over 5 min, at which point the flask was cooled in an ice-water bath. When the internal temperature dropped to 18° C., the cooling bath was removed. Stirred at rt for 1.5 h (incomplete conversion by TLC). An additional 9.5 g of solid NaCN (0.194 mol) was then added, stirred at rt for 2 h (complete conversion by TLC).

The brown reaction mixture was transferred into a 3 L separatory funnel, decanting from ˜5 g of solid NaCN, which had remained undissolved at the bottom of the flask. The solution was combined with water (1 L) and extracted with CH₂Cl₂ (1 L+400 mL+400 mL). The product assay (HPLC) in the three organic phases was, respectively, 76%, 4%, and 0.6%. The product loss in the aq phase after the third extraction was 0.3%.

The first and second organic phases (total assay 80%) were combined, filtered through a short plug of silica (˜100 g silica), and the filtrate was concentrated to 47.1 g weight (66% wt % purity by HPLC). A 20.6 g aliquot of the dark brown oil was distilled at 1 mm Hg and 60-70° C. to provide 12.7 g of 16 as a light yellow liquid (94 wt % purity, 66% yield based on the aliquot).

### Step 3—Enzymatic Reduction:

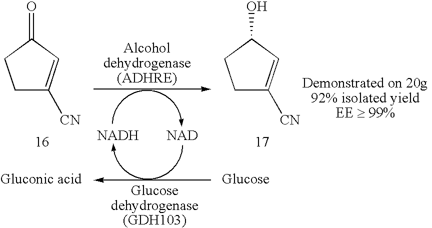

### Procedure:

To a potassium dibasic buffer (0.1 M, pH 7.0, 1 L), glucose (100 g), and nicotinamide adenine dinucleotide (NAD, 4 g) was added which reduced the buffer pH to 6.7. The enzymes were added to the buffer: alcohol dehydrogenase RE (1 g, 37 KU), glucose dehydrogenase 103 (1 g, 67 KU). Two 500 mL reactors were used for the 1 L reaction at temperature of 35° C. and agitation 400 rpm. Substrate 16 (20 g, 0.19 mol) was added directly, 10 g, to each reactor. The pH was controlled at 6.5 using 2.5 M potassium carbonate. Substrate 16 is known to be labile at pH>8.0 so contact with the base was minimized by above surface addition and using a weak base (2.5 M potassium carbonate) instead of the usual 2 N NaOH. Reaction was aged for 20 hours at which point conversion reached greater than 95%. The conversion can be easily monitored by the base consumption to control the pH change from the formation of gluconic acid. The formation of gluconic acid from the cofactor recycling was directly proportional to amount of allylic alcohol product formed. Reaction was extracted by either ethyl acetate or isopropyl alcohol (2 volume extractions) followed by vacuum concentration. Overall yield of 17 was 92% with 1% loss from extraction and <2% residual enone. The 5% mass balance loss was decomposition of 16 under the reaction conditions.

### Step 4—Acylation:

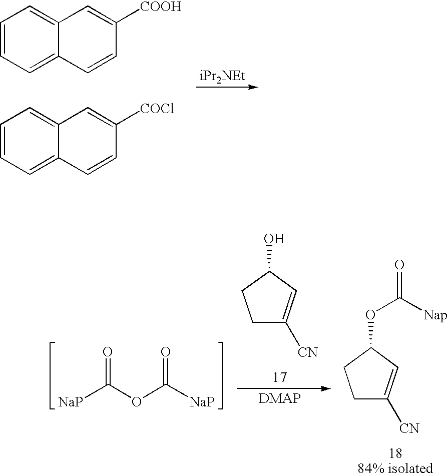

### Procedure:

A 1 L, 1 neck round bottom flask with a magnetic stir bar and nitrogen inlet was charged with 2-naphthoic acid (20.98 g, 122 mmol) and 2-naphthoyl chloride (23.49 g, 122 mmol) and dichloromethane (135 mL). The flask was cooled to an internal temperature of 0° C. Diisopropylethyl amine (76 mL, 4361 mmol) was added while maintaining the internal temperature <5° C. The resulting cloudy, brown solution was warmed to room temperature and stirred for 30 min. (S)-1-Cyano-1-cyclopentene-3-ol (17, 9.50 g, 87.1 mmol) and DMAP (1.07 g, 8.71 mmol) were dissolved in dichloromethane (50 mL). This solution was added to the reaction mixture in one portion and was stirred for 3 hr at room temperature. Water (8.50 mL, 472 mmol) was added and the reaction was stirred for 90 min at room temperature. The reaction was diluted with MTBE (400 mL) and washed with saturated NaHCO₃ (2×400 mL). The organic layer was then washed with H₂O (400 mL), 1M HCl (400 mL) and H₂O (4×400 mL). Total aqueous losses were 1.5%. The dark organic layer was stirred with DARCO KB-B (5.7 g) for 3 h. The solution was filtered through a pad of Solka Floc and concentrated in vacuo at 40° C. to afford a pale yellow solid. The solid was dissolved in MTBE (300 mL) and solvent switched in vacuo at 40° C. to heptane at constant volume. The solid was filtered and washed with heptane to provide 19.24 g of 18 as a pale yellow solid (100.0 wt %, 84% isolated yield). A 3.4% loss was incurred in the mother liquor.

### Step 5—Pd-Catalyzed Etherification:

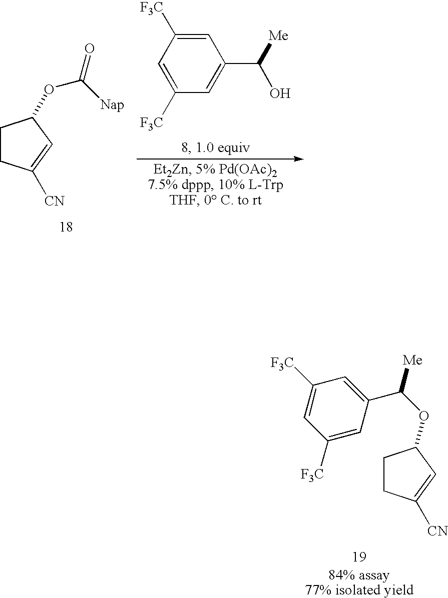

### Procedure:

A 500 mL 3-neck round bottom flask equipped with a thermocouple and nitrogen inlet adapter was evacuated and backfilled with nitrogen. The flask was then carefully charged with alcohol 8 (31.0 g, 120 mmol, 1.0 equiv) minimizing exposure to air, sealed with a septum, and charged with THF (150 mL) via syringe. The solution was cooled in an ice bath to +5° C. 1.0M Et₂Zn in hexane (63 mL, 63 mmol) was added, which resulted in a moderate exotherm to 13° C. The solution was stirred in the ice bath for 30 min, and then nitrile 18 (31.6 g, 120 mmol), Pd(OAc)₂ (1.35 g, 6.01 mmol, 5 mol %), 1,3-bis(diphenylphosphino)propane (3.71 g, 9.00 mmol, 7.5 mol %), and L-tryptophan (2.45 g, 12.0 mmol, 10 mol %) were added to the reaction mixture as solids, taking care to minimize the exposure to air. After 15 min, the ice bath was removed and the reaction mixture was allowed to reach room temperature. After 1 h, a mildly exothermic reaction started and the internal temperature reached 27° C. (no external cooling was applied). HPLC analysis after additional 2 h indicated complete conversion of 18. The thin suspension was transferred into a 1 L flask, and 15 g of Solka Floc was added followed by MTBE (300 mL) while vigorously stirring. The suspension was stirred for 30 min, filtered through Solka Floc, the filtrate was combined with dichloromethane (200 mL), washed with 1M aq HCl (2×500 mL), water (500 mL), 5% aq Na₂CO₃ (3×500 mL), and water (2×500 mL). HPLC analysis of the organic phase revealed 84% assay yield of 19. Darco KB-B (15 g) was added to the organic phase, and the suspension was stirred at room temperature for 18 h, then filtered through Solka Floc. The nearly colorless filtrate was concentrated to an oil to provide 39.78 g of a light tan oil containing 81 wt % 19 (77% isolated yield), 14 wt % of alcohol 8, and 0.7% of ethyl ether 30.

### Step 6—Methyl Ketone Formation:

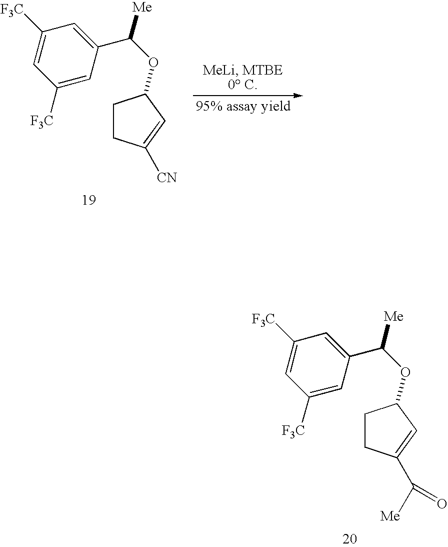

### Procedure:

To a 3-necked, 1 L round bottom flask equipped with nitrogen inlet, thermocouple, and magnetic stirrer was added MTBE (530 mL). The solution was cooled to 0° C., and a solution of MeLi (1.6 M in Et₂O, 123 mL, 0.196 mol) was added. A solution of 19 (41.33 g, 83 wt %, 34.3 g assay, 0.098 mol) in 160 mL MTBE was added via addition funnel, keeping the temperature of the batch below 5° C. (tₘₐₓ=4° C.). Upon complete addition, the batch was aged for 1 h at 0° C., cooled to −70° C., then charged with trifluoroacetic acid (24 mL, 0.32 mol) in one portion, which resulted in a temperature increase to −40° C. A solution of 10% H₃PO₄ (250 mL) was added, and the resulting mixture was warmed to room temperature and aged for 30 minutes. The biphasic mixture was added into 200 mL MTBE and 200 mL 10% H₃PO₄. The organic phase was washed with 500 mL water, then with 500 mL 1 M Na₂CO₃, then with 500 mL water, then with 250 mL water. The organic phase was dried over sodium sulfate and assayed to show 34.5 g of 20 (96% assay yield, 87.9 LCAP, 10.6% benzyl alcohol 8 from Pd-etherification).

The sodium carbonate wash is critical to the success of the subsequent conjugate addition. In the absence of this wash, only ˜20% conversion was observed. It is unknown how many of the water washes are necessary.

### Step 7—Conjugate Addition/Isomerization:

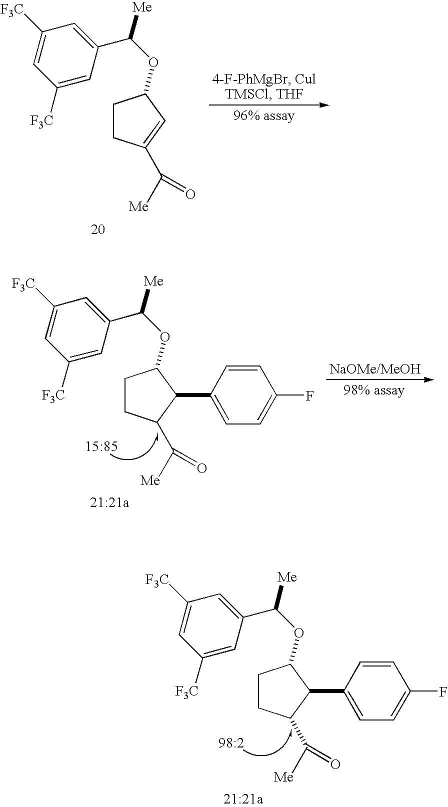

### Procedure:

To a 3-necked, 1 L round bottom flask equipped with a nitrogen inlet, thermocouple, and magnetic stirrer was added CuI (8.8 g, 0.046 mol). The flask was purged with nitrogen for 1 hour. The flask was charged with THF (255 ml), and the slurry was cooled to 0° C. A solution of grignard (2.0 M in Et₂O, 68.2 mL, 0.136 mol) was added at 0° C., keeping the temperature below 10° C. After aging for 30 minutes, the mixture was cooled to −70° C., and TMSCl (32.0 mL, 0.252 mol) was added followed by a solution of 20 (41.4 g, 79 wt %, 32.7 g assay, 0.089 mol) in THF (180 mL+70 mL wash) at 0° C. The temperature of the reaction was not allowed to exceed 40° C. during this addition. The reaction was allowed to warm from −40° C. to −20° C. over the course of 1 hour, which showed 98.4% conversion by HPLC analysis. The reaction was then warmed to 0° C. and aged for 1 hour, which showed complete conversion. The mixture was quenched with 1 M HCl (255 mL) at 0° C., which resulted in an exotherm to 27° C. The mixture was aged at room temperature for 1 hour, transferred into MTBE (500 mL), and separated the layers. The organic layer was washed with 1 M HCl (500 mL) and then water (500 mL). At this point a solid precipitated out of solution. The mixture was filtered over a bed of solka floc (wetted with MTBE), and washed the bed with 250 mL MTBE. The layers were separated and washed the organic layer with 250 mL water. The organic layer was dried over sodium sulfate, concentrated to 600 mL, and assayed to show 39.52 g assay of 21 (15:85 mixture of diastereomers).

The crude solution of the product in MTBE was concentrated to an oil and diluted with 400 mL MeOH into a 3-necked, 1 L round bottom flask equipped with a nitrogen inlet, thermocouple, and magnetic stirrer. The flask was submerged in a 20° C. water bath, and NaOMe (25 wt % in MeOH, 10 mL, 0.044 mol) was added to the reaction slowly, keeping the temperature below 25° C. After an age of only 1 hour, the ratio of diastereomers had increased from 15:85 to 98:2. The reaction was cooled to 0° C., diluted with 600 mL heptane, and quenched with 1M HCl (500 mL). The biphasic mixture was allowed to settle, and the organic layer was separated (2% loss to aqueous). The organic layer was washed twice with water (250 mL), dried with sodium sulfate, and concentrated to yield 21 as an oil (47.9 g, 80.8 wt % of 21, 98% assay), 79.8 LCAP, 10.5 area % benzyl alcohol 8).

### Step 8—Iodoketone Formation:

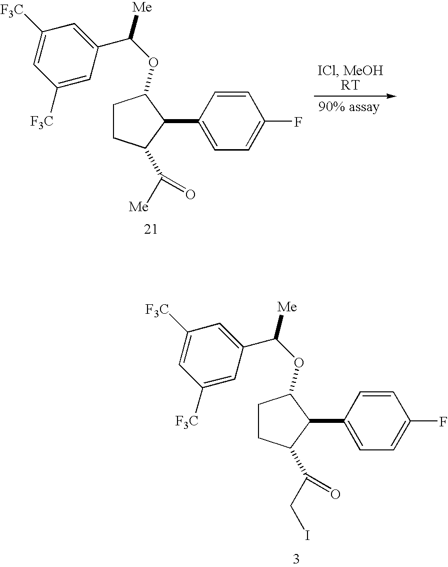

### Procedure:

To a 3-necked, 500 mL round bottom flask equipped with nitrogen inlet, thermocouple, and magnetic stirrer was added 21 (24.6 g, 80.8 wt %, 19.8 g assay, 0.0429 mol) in methanol (230 mL). This solution was submerged in a 20° C. water bath, and ICl (1.0 M in CH₂Cl₂, 77.5 mL, 0.0775 mol) was added dropwise over 20 min, keeping the temperature below 25° C. The reaction was aged for 2 hours at room temperature at which point complete conversion was observed by HPLC analysis. The mixture was quenched into MTBE (250 mL) and 10% Na₂S₂O₃/5% NaHCO₃ (250 mL) at −10° C. This mixture was further diluted with 200 mL MTBE and 200 mL 10% Na₂S₂O₃/5% NaHCO₃, then separated the layers. The organic layer was washed twice with water (300 mL), dried using sodium sulfate, and assayed to show 22.0 g of iodoketone 3 (87% yield). The organic solution was concentrated to a solid, and diluted with methanol (170 mL). The vessel was seeded with 50 mg of iodoketone, and water (37.5 mL) was added dropwise to the reaction mixture over 2 hours. The mixture was aged overnight, assayed the supernatent (5.9 mg/mL), filtered, and washed with 70 mL of 70:30 MeOH:H₂O to yield 22.4 g of 3 as a white solid (93 wt % iodoketone, 20.8 assay, 83%; 97.3 LCAP, 0.9% Cl-ketone, 1.4% ketone isomer).

### Salts

Various crystalline salts of the compound of Formula Ia

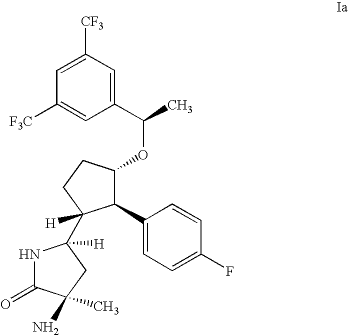

were made and evaluated. Physicochemical data for these salts are shown in the following table.

| | | | | Solid State | Solid State | |
|---|---|---|---|---|---|---|
| | Crystalline | Melting T | Hygroscopicity | Chemical | Physical | Process |
| Salt | form | (° C.) | (25° C.) | Stability | Stability² | ability |
| L-tartrate | Hydrate | 205 | Hygroscopic | N/A | Stable | N/A |
| Succinate | Hemihydrate | 187 | Not | Amide | Stable | N/A |
| | | | Hygroscopic | adduct | | |
| Besylate | Anhydrous | 271 | Slightly | Stable | Stable³ | N/A |
| | Form II | | Hygroscopic¹ | | | |
| Besylate | Anhydrous | 273 | Not | Stable | Stable³ | N/A |
| | Form I | | Hygroscopic | | | |

¹Hygroscopic at >75% RH, depending on crystallization conditions
²No form conversion after 1 week at 40° C./ambient RH, 40° C./75% RH, and 80° C.
³Both forms convert rapidly to Type C hydrate in aqueous solution. Type C hydrate converts to Form III when isolated; this form is less stable than Form II at <140° C.

The anhydrous form I crystalline form of the besylate salt is both physically and chemically stable, is more thermodynamically stable than form II and has been consistently non-hygroscopic.

X-ray powder diffraction studies are widely used to characterize molecular structures, crystallinity, and polymorphism. The X-ray powder diffraction pattern of the crystalline anhydrous Form I of the besylate salt was generated on a Philips Analytical X'Pert PRO X-ray Diffraction System with PW3040/60 console. A PW3373/00 ceramic Cu LEF X-ray tube K-Alpha radiation was used as the source.

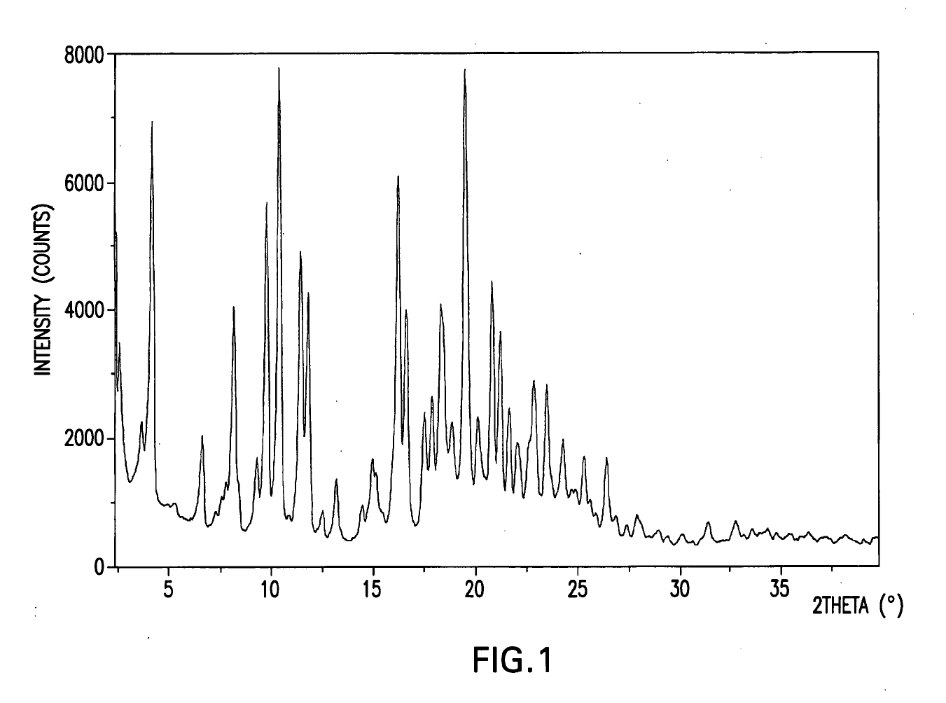

FIG. 1 shows the X-ray diffraction pattern for the crystalline anhydrous Form I of the besylate salt. The anhydrous Form I exhibited characteristic diffraction peaks corresponding to d-spacings of 21.2, 9.1, and 8.5 angstroms. The anhydrous Form I was further characterized by the d-spacings of 13.5, 10.9 and 5.5 angstroms. The anhydrous Form I was even further characterized by the d-spacings of 4.5, 4.3, and 4.2 angstroms.

DSC data were acquired at a heating rate of 10° C./min, under nitrogen atmosphere in a closed pan using TA Instruments DSC 2910 or equivalent instrumentation.

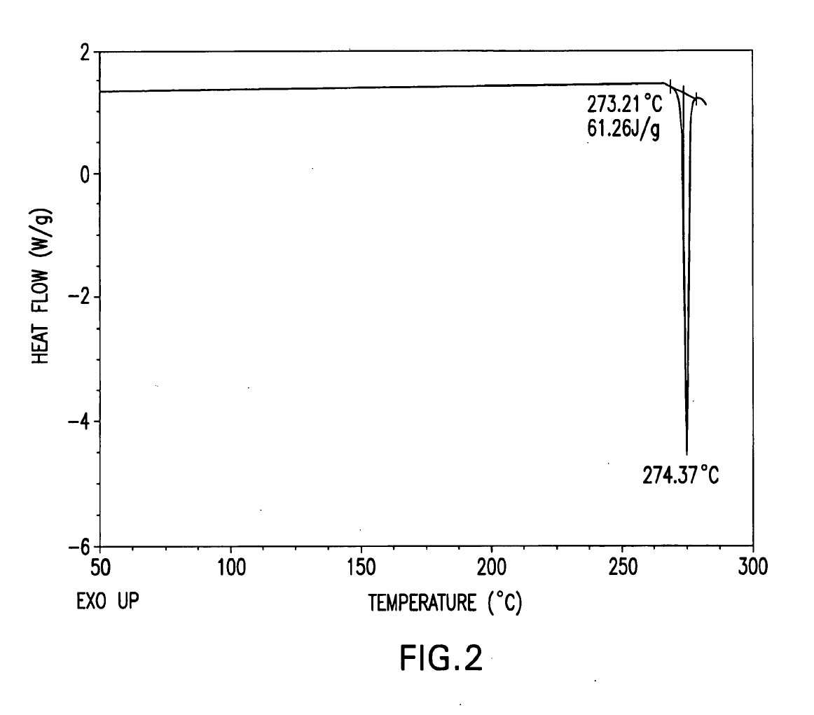

FIG. 2 shows the differential calorimetry scan for the crystalline anhydrous Form I of the besylate salt. The crystalline anhydrous Form I exhibited an endotherm due to melting with an onset temperature of 273.2° C., the peak temperature is 274.4° C. and the enthalpy change is 61.3 J/g.

What is claimed is:

1. A process of making a compound of Formula I 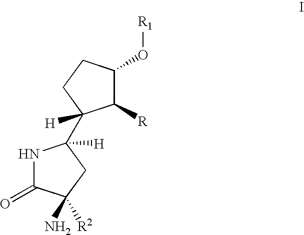
or a pharmaceutically acceptable salt thereof, wherein:
R² is selected from the group consisting of:
(1) hydrogen, and
(2) C₁₋₆alkyl;
R is selected from the group consisting of:
(1) phenyl, unsubstituted or substituted with one or more of R¹¹, R¹² and R¹³;
(2) C₁₋₈ alkyl, unsubstituted or substituted with one or more of the substituents selected from:
(a) hydroxy,
(b) oxo,
(c) C₁₋₆ alkoxy,
(d) phenyl-C₁₋₃ alkoxy,
(e) phenyl,
(f) —CN,
(g) halo,
(h) —NR⁹R¹⁰, wherein R⁹ and R¹⁰ are independently selected from:
(1) hydrogen,
(2) C₁₋₆ alkyl,
(3) hydroxy-C₁₋₆ alkyl, and
(4) phenyl,
(i) —NR⁹COR¹⁰, wherein R⁹ and R¹⁰ are as defined above,
(j) —NR⁹CO₂R¹⁰, wherein R⁹ and R¹⁰ are as defined above,
(k) —CONR⁹R¹⁰, wherein R⁹ and R¹⁰ are as defined above,
(l) —COR⁹, wherein R⁹ is as defined above, and
(m) —CO₂R⁹, wherein R⁹ is as defined above;
(3) C₂₋₆ alkenyl, unsubstituted or substituted with one or more of the substituent(s) selected from:
(a) hydroxy,
(b) oxo,
(c) C₁₋₆ alkoxy,
is (d) phenyl-C₁₋₃ alkoxy,
(e) phenyl,
(f) —CN,
(g) halo,
(h) —CONR⁹R¹⁰ wherein R⁹ and R¹⁰ are as defined above,
(i) —COR⁹ wherein R⁹ is as defined above,
(j) —CO₂R⁹, wherein R⁹ is as defined above;
(4) heterocycle, wherein the heterocycle is selected from the group consisting of:
(A) benzimidazolyl,
(B) benzofuranyl,
(C) benzothiophenyl,
(D) benzoxazolyl,
(E) furanyl,
(F) imidazolyl,
(G) indolyl,
(H) isooxazolyl,
(I) isothiazolyl,
(J) oxadiazolyl,
(K) oxazolyl,
(L) pyrazinyl,
(M) pyrazolyl,
(N) pyridyl,
(O) pyrimidyl,
(P) pyrrolyl,
(Q) quinolyl,
(R) tetrazolyl,
(S) thiadiazolyl,
(T) thiazolyl,
(U) thienyl,
(V) triazolyl,
(W) azetidinyl,
(X) 1,4-dioxanyl,
(Y) hexahydroazepinyl,
(Z) piperazinyl,
(AA) piperidinyl,
(AB) pyrrolidinyl,
(AC) tetrahydrofuranyl, and
(AD) tetrahydrothienyl,
and wherein the heterocycle is unsubstituted or substituted with one or more substituent(s) selected from:
(i) C₁₋₆ alkyl, unsubstituted or substituted with halo, —CF₃, —OCH₃, or phenyl,
(ii) C₁₋₆ alkoxy,
(iii) oxo,
(iv) hydroxy,
(v) thioxo,
(vi) —SR⁹, wherein R⁹ is as defined above,
(vii) halo,
(viii) cyano,
(ix) phenyl,
(x) trifluoromethyl,
(xi) —(CH₂)ₘ—NR⁹R¹⁰, wherein m is 0, 1 or 2, and R⁹ and R¹⁰ are as defined above,
(xii) —NR⁹COR¹⁰, wherein R⁹ and R¹⁰ are as defined above,
(xiii) —CONR⁹R¹⁰, wherein R⁹ and R¹⁰ are as defined above,
(xiv) —CO₂R⁹, wherein R⁹ is as defined above, and
(xv) —(CH₂)ₘ—OR⁹, wherein m and R⁹ are as defined above;
R¹ is selected from the group consisting of:
(1) 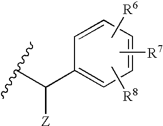
(2) —C₁₋₈ alkyl, wherein alkyl is unsubstituted or substituted with one or more of the substituents selected from:
(a) hydroxy,
(b) oxo,
(c) C₁₋₆ alkoxy,
(d) phenyl-C₁₋₃ alkoxy,
(e) phenyl,
(f) —CN,
(g) halo,
(h) —NR⁹R¹⁰, wherein R⁹ and R¹⁰ are as defined above,
(i) —NR⁹COR¹⁰, wherein R⁹ and R¹⁰ are as defined above,
(j) —NR⁹CO₂R¹⁰, wherein R⁹ and R¹⁰ are as defined above,
(k) —CONR⁹R¹⁰, wherein R⁹ and R¹⁰ are as defined above,
(l) —COR⁹, wherein R⁹ is as defined above, and
(m) —CO₂R⁹, wherein R⁹ is as defined above;
(3) —C₂₋₆ alkenyl, wherein alkenyl is unsubstituted or substituted with one or more of the substituent(s) selected from:
(a) hydroxy,
(b) oxo,
(c) C₁₋₆ alkoxy,
(d) phenyl-C₁₋₃ alkoxy,
(e) phenyl,
(f) —CN,
(g) halo,
(h) —CONR⁹R¹⁰ wherein R⁹ and R¹⁰ are as defined above,
(i) —COR⁹ wherein R⁹ is as defined above,
(j) —CO₂R⁹, wherein R⁹ is as defined above;
(4) —(CO)-phenyl, wherein the phenyl is unsubstituted or substituted with one or more of R⁶, R⁷ and R⁸;
R⁶, R⁷ and R⁸ are independently selected from the group consisting of:
(1) hydrogen;
(2) C₁₋₆ alkyl, unsubstituted or substituted with one or more of the substituents selected from:
(a) hydroxy,
(b) oxo,
(c) C₁₋₆ alkoxy,
(d) phenyl-C₁₋₃ alkoxy,
(e) phenyl,
(f) —CN,
(g) halo,
(h) —NR⁹R¹⁰, wherein R⁹ and R¹⁰ are as defined above,
(i) —NR⁹COR¹⁰, wherein R⁹ and R¹⁰ are as defined above,
(j) —NR⁹CO₂R¹⁰, wherein R⁹ and R¹⁰ are as defined above,
(k) —CONR⁹R¹⁰, wherein R⁹ and R¹⁰ are as defined above,
(l) —COR⁹, wherein R⁹ is as defined above, and
(m) —CO₂R⁹, wherein R⁹ is as defined above;
(3) C₂₋₆ alkenyl, unsubstituted or substituted with one or more of the substituent(s) selected from:
(a) hydroxy,
(b) oxo,
(c) C₁₋₆ alkoxy,
(d) phenyl-C₁₋₃ alkoxy,
(e) phenyl,
(f) —CN,
(g) halo,
(h) —CONR⁹R¹⁰ wherein R⁹ and R¹⁰ are as defined above,
(i) —COR⁹ wherein R⁹ is as defined above,
(j) —CO₂R⁹, wherein R⁹ is as defined above;
(4) C₂₋₆ alkynyl;
(5) phenyl, unsubstituted or substituted with one or more of the substituent(s) selected from:
(a) hydroxy,
(b) C₁₋₆ alkoxy,
(c) C₁₋₆alkyl,
(d) C₂₋₅ alkenyl,
(e) halo,
(f) —CN,
(g) —NO₂,
(h) —CF₃,
(i) —(CH₂)ₘ—NR⁹R¹⁰, wherein m, R⁹ and R¹⁰ are as defined above,
(j) —NR⁹COR¹⁰, wherein R⁹ and R¹⁰ are as defined above,
(k) —NR⁹CO₂R¹⁰, wherein R⁹ and R¹⁰ are as defined above,
(l) —CONR⁹R¹⁰, wherein R⁹ and R¹⁰ are as defined above,
(m) —CO₂NR⁹R¹⁰, wherein R⁹ and R¹⁰ are as defined above,
(n) —COR⁹, wherein R⁹ is as defined above;
(o) —CO₂R⁹, wherein R⁹ is as defined above;
(6) halo,
(7) —CN,
(8) —CF₃,
(9) —NO₂,
(10) —SR¹⁴, wherein R¹⁴ is hydrogen or C₁₋₅alkyl,
(11) —SOR¹⁴, wherein R¹⁴ is as defined above,
(12) —SO₂R¹⁴, wherein R¹⁴ is as defined above,
(13) NR⁹COR¹⁰, wherein R⁹ and R¹⁰ are as defined above,
(14) CONR⁹COR¹⁰, wherein R⁹ and R¹⁰ are as defined above,
(15) NR⁹R¹⁰, wherein R⁹ and R¹⁰ are as defined above,
(16) NR⁹CO₂R¹⁰, wherein R⁹ and R¹⁰ are as defined above,
(17) hydroxy,
(18) C₁₋₆alkoxy,
(19) COR⁹, wherein R⁹ is as defined above,
(20) CO₂R⁹, wherein R⁹ is as defined above,
(21) 2-pyridyl,
(22) 3-pyridyl,
(23) 4-pyridyl,
(24) 5-tetrazolyl,
(25) 2-oxazolyl, and
(26) 2-thiazolyl;
R¹¹, R¹² and R¹³ are independently selected from the definitions of R⁶, R⁷ and R⁸; and
Z is selected from:
(1) hydrogen,
(2) C₁₋₆ alkyl, and
(3) hydroxyl;
comprising reacting a compound of Formula A 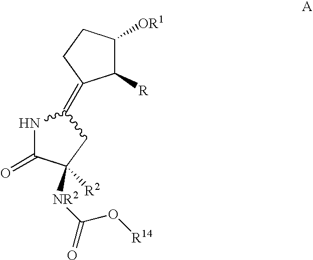
wherein R¹⁴ is selected from R⁶, with a reducing agent under acidic conditions to yield a compound of Formula B 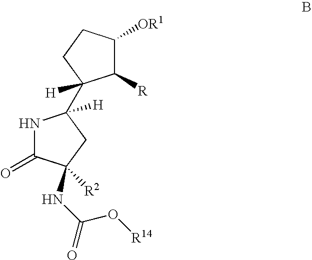
and reacting a compound of Formula B with a strong acid to yield a compound of Formula I, and optionally subsequently forming a pharmaceutically acceptable salt of the compound of Formula I by reacting the compound of Formula I with the corresponding acid of the salt to form the pharmaceutically acceptable salt of the compound of Formula I.

2. The process according to claim 1, further comprising making the compound of Formula A by reacting a compound of Formula C 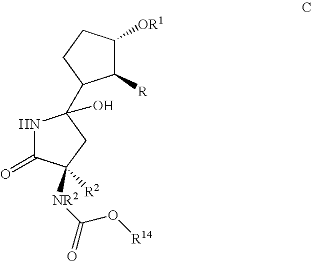
with an acid to make a compound of Formula A.

3. The process according to claim 2, further comprising making the compound of Formula C by reacting a compound of Formula D 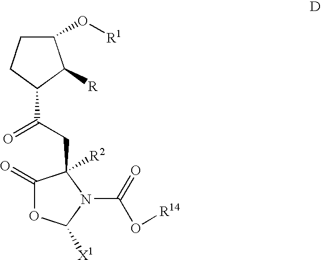
wherein X¹ is C₁₋₆alkyl or phenyl, with ammonia or a salt thereof to yield a compound of Formula C.

4. The process according to claim 3, further comprising making the compound of Formula D by reacting a compound of Formula E 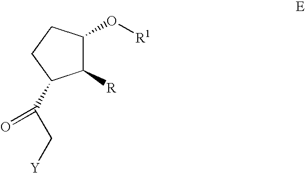
wherein Y is a halogen, with a compound of Formula F 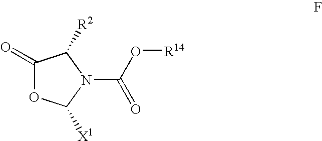
and a metal amide of Formula M¹N(R¹⁵)₂ or M¹N(Si(R¹⁵)₃)₂, wherein M¹ is Li, Na, K or Mg, and each R¹⁵ is independently selected from C₁₋₄alkyl, in a first aprotic organic solvent to yield a compound of Formula D.

5. The process according to claim 4, further comprising making the compound of Formula E by reacting the compound of Formula G 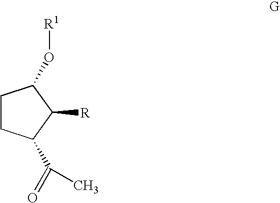
with a halogenating agent to yield a compound of Formula E.

6. The process according to claim 5, further comprising making the compound of Formula G by reacting the compound of Formula H 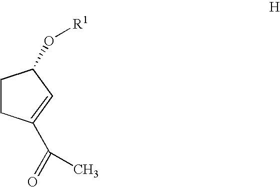
with R-M², wherein M² is a metal, in the presence of a first transition metal catalyst and a Lewis acid, to yield a compound of Formula G.

7. The process according to claim 6, further comprising making the compound of Formula H by reacting a compound of Formula J 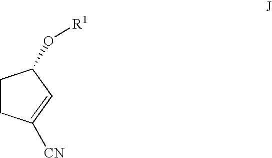
with CH₃Li in tert-butyl methyl ether to yield a compound of Formula H.

8. The process according to claim 7, further comprising making the compound of Formula J by reacting a compound of Formula K 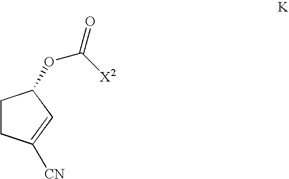
wherein X² is selected from R, with R¹—OH in the presence of a second transition metal catalyst catalyst, a ligand and a zinc additive to yield a compound of Formula J.

9. The process according to claim 8, further comprising making the compound of Formula K by enzymatically reducing the compound of Formula L 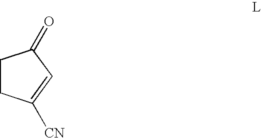
and subsequently reacting the product with X²—COCl to yield a compound of Formula K.

10. A process according to claim 9, further comprising making the compound of Formula L by reacting a compound of Formula M 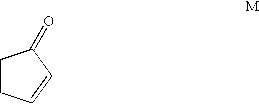
with a brominating agent followed by reaction with a cyanating agent in the presence of a buffer to yield a compound of Formula L.

11. A process according to claim 4, wherein Y is I and further comprising making the compound of Formula E by reacting a compound of Formula N 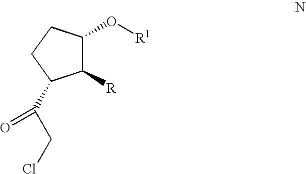
with M³-I, wherein M³ is Li, Na or K, to yield a compound of Formula E.

12. A process according to claim 11, further comprising making the compound of Formula N by reacting a compound of Formula O 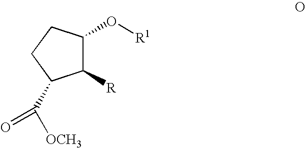
with ClCH₂CO₂H, ClCH₂I, or ClCH₂Br and a metal amide of Formula M⁴N(R¹⁶)2 or M⁴N(Si(R¹⁶)₃)₂, wherein M⁴ is Li, Na, K, or Mg, and each R¹⁶ is independently selected from C₁₋₄alkyl in a second aprotic organic solvent at a temperature range of about −20° C. to about 40° C. to yield a compound of Formula N.

13. The process according to claim 12, further comprising making the compound of Formula O by reacting a compound of Formula P 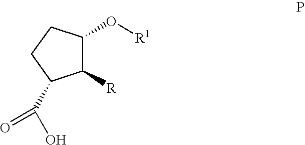
or a triethylamine salt thereof, with a methylating agent to yield a compound of Formula O.

14. A process according to claim 13, further comprising making the compound of Formula P by reacting a compound of Formula Q 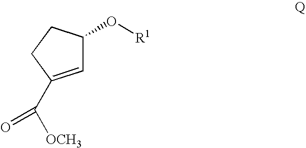
with R-M⁵, wherein M⁵ is M² is a metal, in the presence of a first transition metal catalyst and a Lewis acid, and optionally followed by triethylamide to form the salt, to yield the compound of Formula P or the triethylamine salt thereof.

15. The process according to claim 14 wherein the compound of Formula Q is made by reacting a compound of Formula R 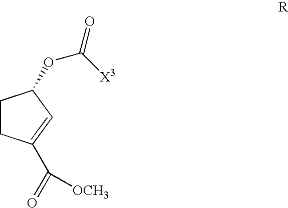
wherein X³ is selected from R, with R¹—OH in the presence of a second transition metal catalyst, a ligand and a zinc additive to yield a compound of Formula Q.

16. The process according to claim 15, further comprising making the compound of Formula R by enzymatically reducing the compound of Formula S 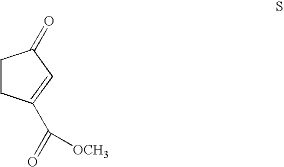
and subsequently reacting the product with X³—COCl to yield a compound of Formula R.

17. The process according to claim 16, further comprising making the compound of Formula S by reacting a compound of Formula T 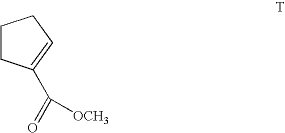
with an oxidizing agent to yield a compound of Formula S.

18. The process according to claim 1 wherein R¹ is

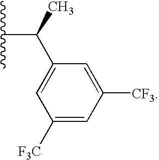

19. The process according to claim 1 wherein R is

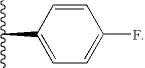

20. The process according to claim 1 wherein R² is methyl.

21. The process according to claim 1 herein the compound of Formula I is a compound of Formula Ia

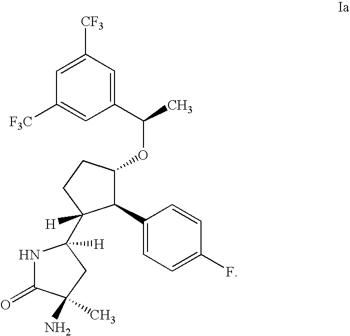

22. The process according to claim 21 wherein the compound of Formula Ia is a pharmaceutically acceptable salt.

23. The process according to claim 22 wherein the pharmaceutically acceptable salt is the benzenesulfonate salt and the corresponding acid of the salt is benzenesulfonic acid.

24. The benzenesulfonate salt of a compound of Formula Ia:

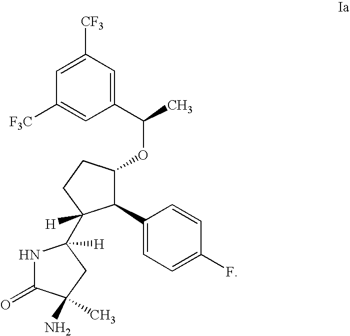

25. An anhydrous crystalline form of the benzenesulfonate salt of claim 24 designated Form I and exhibiting characteristic diffraction peaks corresponding to d-spacings of about 21.2, 9.1, and 8.5 angstroms.

26. The anhydrous crystalline form designated Form I of claim 25 further characterized by the d-spacings of 13.5, 10.9 and 5.5 angstroms.

27. The anhydrous crystalline form designated Form I of claim 26 further characterized by the d-spacings of 4.5, 4.3, and 4.2 angstroms.
